(19) United States
(12) Patent Application Publication
(10) Pub. No.: US 2002/0031509 A1
(43) Pub. Date: Mar. 14, 2002

(54) Pharmaceutical composition and method for its manufacture

(76) Inventors: Bjorn Ortenheim, Edsbro (SE); Martin Shalling, Stockholm (SE); Fabio Sanchez, Stockholm (SE); Catharina Lavebratt Holmquist, Sollentuna (SE)

Correspondence Address: BIRCH STEWART KOLASCH & BIRCH, PO BOX 747, FALLS CHURCH, VA, 22040-0747, US

(21) Appl. No.: 09/747,923

(22) Filed: Dec. 27, 2000

Related U.S. Application Data
(60) Provisional application No. 60/173,290, filed on Dec. 28, 1999.

(30) Foreign Application Priority Data
Oct. 6, 2000 (SE) 0003619-4

(51) Int. Cl. A61K 38/48; A61K 35/58

(52) U.S. Cl. 424/94.67; 424/542

(57) ABSTRACT

The present invention relates to a pharmaceutical composition comprising an antimicrobially effective amount of at least one snake venom obtainable from a snake selected from the group of snakes consisting of: Naja kaouthia, Bungarus fasciatus, Ophiophagus hannah, Bungarus candidus, Lapemis hardwickii, Hydrophis cyanocinctus, Enhydrina schistosa, Aipysurus eydouxii, Bothrops atrox, Lachesis muta, Bitis arietans, Trimersurus albolabris or combinations thereof; and a pharmaceutically acceptable carrier. The composition may further comprise one or more fractions or one or more components of the above venoms. The present invention also relates to medical use of snake venoms and use of said venoms for the manufacture of medicaments for the prevention, management or treatment of bacterial, fungal, protozoan or viral diseases. Also the present invention also relates to a composition above further including a plant extract.

The present invention relates to a pharmaceutical composition comprising an antimicrobially effective amount of at least one snake venom obtainable from a snake selected from the group of snakes consisting of: Naja Kaouthia, Bungarus fasciatus, Ophiophagus hannah, Bungarus candidas, Lapemis hardwickii, Hydrophis cyanocinctus, Enhydrina schistosa, Aipysurus eydouxii, Bothrops atrox, Lachesis muta, Bitis arietans, Trimersurus albolabris or combinations thereof and a pharmaceutically acceptable carrier. The present invention also relates to medical use of snake venoms and use of said venoms for the manufacture of medicaments for the prevention, management or treatment of bacterial, fungal, protozoan or viral diseases. Also the present invention also relates to a composition above further including a plant extract.

### BACKGROUND

Snake venoms are complex mixtures of substances primarily designed to paralyse and digest prey. It is known that such venoms may have neurotoxic, haemotoxic and proteolytic properties. Substances that affect the peripheral and central nervous system and muscular system, have been isolated from such venoms. A substance that affect specific components of the coagulation cascade has been described which have been isolated from Bungarus fasciatus (see Zhang Y. et al, “An activator of blood coagulation factor X from the venom of Bungarus fasciatus”, Toxicon, 1995 October; 33(10): 1277-88). Some of those substances has been desribed for use in clinical as well as basic research situations. One example of them is Captopril which is known to be used medically for lowering the blood pressure. See e.g. “Djurens gifter blir vara mediciner”, L. Thomas, Illustrerad Vetenskap, page 78-81, No:8, 1998, which deals with snake venom from Bothrops jararacas containing said substance. Further an analgesic toxin from Ophiophagus hannah has been discribed in Pu XC et al, “A novel analgesic toxin (hannalgesin) from the venom of king cobra (Ophiophagus hannah)”, Toxicon 1995, November; 33(11);1425-31). New et al describes in U.S. Pat. No. 4,661,346 immunological compositions for eliciting antibodies where use of snake venoms or components thereof is discussed (venoms mentioned are e.g. from Bungarus candidus, Bitis arietans, Bothrops atrox, Trimeresurus albolabris) for e.g. use against viral foot and mouth disease in cattle. However this disclosure appears only to be directed for prophylactic purposes. Further, Lipps et al discloses in U.S. Pat. No. 5,648,339 a herpoxin, a herpes virus inhibitor isolated from Naja kaoutia which is suggested for use against bacterial, viral and fungal diseases. Haast discloses in U.S. Pat. No. 4,341,762 use of snake venoms for treatment of neurological and related disorders. However, treatment of bacterial, fungal or viral infectious diseases with snake venoms or components thereof, from either of Bungarus fasciatus, Ophiophagus hannah, Bungarus candidas, Lapemis hardwickii, Hydrophis cyanocinctus, Enhydrina schistosa, Aipysurus eydouxii, Bothrops atrox, Lachesis muta, Bitis arietans, Trimersurus albolabris has not been described, to our knowledge.

Pharmaceutical compositions for treatment of bacterial or viral infectious diseases are known in the art. E.g. penicillin, aminoglycosides, cephalosporines among others are used when treating bacterial diseases. However the usefulness of such substances is decreasing mainly because of the increasing occurence of resistance to these antibiotics among bacterial strains and not infrequently by the cost and unavailability of them particularly in developing countries in the third world. Viral infections represent a big challenge for clinicians since there is not a single specific substance that can be used to eradicate these infections from an affected.

Thus there is a need for new pharmaceutical compositions for treatment of infectious diseases that can be an alternative for cases of multi-drug resistance or viral infections and which can be available and affordable to communities around the world, and especially in the third world.

### SUMMARY OF THE INVENTION

The present invention solves the above problem by providing new pharmaceutical compositions comprising an antimicrobially effective amount of at least one snake venom obtainable from a snake selected from the group of snakes consisting of: Naja kaouthia, Bungarus fasciatus, Ophiophagus hannah, Bungarus candidus, Lapemis hardwickii, Hydrophis cyanocinctus, Enhydrina schistosa, Aipysurus eydouxii, Bothrops atrox, Lachesis muta, Bitis arietans, Trimersurus albolabris or combinations thereof; and a pharmacetically acceptable carrier; which may be administrated orally or by other routes as well. The compositions according to the present invention may be used for the prevention, management or treatment of bacterial, fungal, protozoan or viral diseases

### DETAILED DESCRIPTION OF THE INVENTION

Snake venoms contain many different components and some of these are toxic to man. For instance, in the common tiger snake venom, Notechis scutatus, there have been at least 6 neurotoxins described. There are several haemotoxins, 2 interfering with activation of the clotting factors and some weak haemorrhagins. Some of the neurotoxins also cause muscle damage. There are also venom toxins which can cause a drop in blood pressure as mentioned above. Most venom components are still uncharacterised.

The snake venoms referred to in the present application are from the order Squamata (Lizards and snakes), suborder Scleroglossa, infraorder Ophidia (serpents), superfamily Xenophidia, comprising the families Viperidae and Elapidae which comprise the snakes referred to in the present application. Members of the family Viperidae are “venomous” snakes. They occur on all continents except Australia and may be found in most ecological habitats from tropical rain forests to deserts and even high mountains. They may be viviparous or oviparous.

Naja kaouthia (Siamese cobra), Bungarus fasciatus (Banded krait), Ophiophagus hannah (King cobra) and Bungarus candidus (Malayan krait) belong to the subfamily Bungarinae, in turn belonging to family Elapidae. The poison of elapids such as Naja kaouthia, Bungarus fasciatus, Ophiophagus hannah and Bungarus candidus influences the human nervous system (i.e. they have neurotoxic properties).

Bothrops atrox, Lachesis muta, Trimersurus albolabris belong to the subfamily Crotalinae (Pit vipers) and Bitis arietans belong to the subfamily Viperinae (Pitless vipers). Both subfamilies belong to the family Viperidae. Often the subfamily Hydrophiidae is included into the family Elapidae, and Lapemis hardwickii (Hardwicke's sea snake), Hydrophis cyanocinctus (Bluebanded sea snake), Enhydrina schistosa. (Beaked sea snake) and Aipysurus eydouxii (White spotted sea snake) are included in subfamily Hydrophiidae. The poisons of vipers and Hydrophiidae such as Lapemis hardwickii, Hydrophis cyanocinctus, Enhydrina schistosa, Aipysurus eydouxii, Bothrops atrox, Lachesis muta, Bitis arietans and Trimersurus albolabris influence (i.e. is toxic to) the human muscular system and they also influence the blood coagulation. The venoms also have proteolytic properties.

As used herein, the term “snake venom” refers to a whole snake venom, one or more fractions from one snake venom, one or more components from one snake venom or combinations thereof. The fractions may preferably be obtainable by a fractionation method according to one preferred embodiment of the present application as set out below. The components may be crude, purified or modified. The components may be obtained from natural sources (including gene modified cells, e.g. bacteria) or they may be synthesized chemically.

As used herein, the term “antimicrobial” refers to antibacterial (which includes antimycobacterial), antiviral, antifungal, antiprotozoan or antinematodal effect. The bacteria, that may give rise to infectious diseases, may be Gram negative, e.g. E. coli, or Gram positive, e.g. Staphylococcus or Bacillus. Other bacteria may be Serratia, Salmonella and Pseudomonas. The viruses, that may give rise to infectious diseases, may be influenza virus, herpes simplex virus, adenovirus, RS virus, HIV, Ebola, or other. One fungus that may give rise to an infectious disease is Candida albicans.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a human or other mammal without the production of undesirable physiological effects such as nausea, dizziness, gastric upset and the like.

As used herein, the expression “plant extract” refers to a plant poison, a mixture of plant poisons, a preparation, an extract or a substance obtainable from the plant family Ranunculaceae with an antimicrobial effect. The preparation and/or extract may have other inactive substances as diluents. Preferably the poison is influencing the human muscular system and is obtained from the plant Aconitum septentrionale, Aconitum karacolicum or Aconitum napellus. The posion may preferably be Aconitine (or isomers thereof) which is an extremely toxic potent central nervous system poison (also known as 16-ethyl-1,16,19-trimethoxy-4-(methoxymethyl)aconitane-3,8,10,11,18-pentol 8-acetate 10-benzoate). Even in extremely small amounts, this potent chemical can inhibit respiration and in larger amounts can lead to complete heart failure. Aconitine may be used for creating models of cardiac arrhythmia. Aconitine may be obtained from Aconitum napellus and Aconitum septentrionale or from other of the family Ranunculaceae. Aconite, i.e. Aconitum napellus, also known as monkshood or Fu-Tzu, is an extremely powerful and potentially toxic herb with a long history of use. In traditional Chinese medicine this herb is considered an effective stimulant for the spleen and kidneys, and is a favourite treatment for malaise, general weakness, poor circulation, cancer, and heart disease. Aconite is also occasionally used in very low doses by modern homeopathic practitioners as a treatment for colds, influenza, rheumatism and congestion. Amorphous aconitine (also known as “mild aconitine”) is a mixture of amorphous alkaloids from Aconitum napellus. Said mixture may include aconitine, mesaconitine, hypaconitine, neopelline, 1-ephederine, sparteine, neoline and napelline. More aconite alkaloids are disclosed in Suzuki, U.S. Pat. No. 5,909,857. Further, anti-viral use of aconite alkaloids is disclosed in said U.S. patent. As used herein, the term “aconitine” refers to aconitine and derivatives or isomers thereof.

According to one preferred embodiment of the present invention a pharmaceutical composition comprising a snake venom obtainable from a snake selected from the group of snakes consisting of: Bungarus fasciatus, Ophiophagus hannah, Bungarus candidus, Bitis arietans or Trimersurus albolabris and a pharmaceutically acceptable carrier is provided. Said compositions may be used for the prevention, management or treatment of bacterial, fungal, protozoan or viral diseases.

According to one preferred embodiment of the present invention a pharmaceutical composition comprising snake venom obtainable from Ophiophagus hannah and a pharmaceutically acceptable carrier is provided. Said composition may be used for the prevention, management or treatment of bacterial, fungal, protozoan or viral diseases, preferably bacterial diseases.

According to one preferred embodiment of the present invention a pharmaceutical composition comprising snake venom obtainable from Bungarus fasciatus and a pharmaceutically acceptable carrier is provided. Said composition may be used for the prevention, management or treatment of bacterial, fungal, protozoan or viral diseases, preferably bacterial diseases.

According to one preferred embodiment of the present invention a pharmaceutical composition comprising snake venom obtainable from Bitis arietans and a pharmaceutically acceptable carrier is provided. Said composition may be used for the prevention, management or treatment of bacterial, fungal, protozoan or viral diseases, preferably bacterial diseases.

According to one preferred embodiment of the present invention a pharmaceutical composition comprising snake venom obtainable from Trimersurus albolabris and a pharmaceutically acceptable carrier is provided. Said composition may be used for the prevention, management or treatment of bacterial, fungal, protozoan or viral diseases, preferably bacterial diseases.

According to one preferred embodiment of the present invention a pharmaceutical composition comprising three snake venoms obtainable from Naja kaouthia, Bungarus fasciatus and Ophiophagus hannah, respectively and a pharmaceutically acceptable carrier is provided. Said composition may be used for the prevention, management or treatment of bacterial, fungal, protozoan or viral diseases, preferably bacterial diseases.

According to one preferred embodiment of the present invention a pharmaceutical composition comprising at least two snake venoms from two different groups, i.e. essentially subfamilies, of snakes, wherein the first venom is influencing the human nervous system (i.e. has essentially neurotoxic properties) and the second venom is influencing the human muscular system (i.e. has essentially proteolytic properties); and a pharmaceutically acceptable carrier is provided. Said composition may be used for the prevention, management or treatment of bacterial, fungal, protozoan or viral diseases, preferably bacterial diseases.

According to the preferred embodiment above of the present invention a pharmaceutical composition comprising at least two snake venoms from two different groups of snakes, wherein the first venom is obtainable from a snake selected from the group of snakes consisting of: Naja kaouthia, Bungarus fasciatus, Ophiophagus hannah and Bungarus candidus; and the second venom is obtainable from a snake selected from the group of snakes consisting of: Lapemis hardwickii, Hydrophis cyanocinctus, Enhydrina schistosa, Aipysurus eydouxii, Bothrops atrox, Lachesis muta, Bitis arietans or Trimersurus albolabris; and a pharmaceutically acceptable carrier is provided. Said composition may be used for the prevention, management or treatment of bacterial, fungal, protozoan or viral diseases, preferably bacterial.

According to one preferred embodiment of the present invention a pharmaceutical composition comprising at least two snake venoms from two different groups of snakes, wherein the first venom is obtainable from Ophiophagus hannah and the second venom is obtainable from Bitis arietans or Trimersurus albolabris; and a pharmaceutically acceptable carrier is provided. Said composition may be used for the prevention, management or treatment of bacterial, fungal, protozoan or viral diseases, preferably bacterial.

According to one preferred embodiment of the present invention a pharmaceutical composition comprising at least one snake venom obtainable from a snake selected from the group of snakes consisting of: Naja kaouthia, Bungarus fasciatus, Ophiophagus hannah, Bungarus candidus, Lapemis hardwickii, Hydrophis cyanocinctus, Enhydrina schistosa, Aipysurus eydouxii, Bothrops atrox, Lachesis muta, Bitis arietans, Trimersurus albolabris or combinations thereof, preferably one influencing the muscular system, and at least one plant extract; and a pharmaceutically acceptable carrier is provided. Preferably this snake venom is from Bitis arietans or Trimersurus albolabris. Said composition may be used for the prevention, management or treatment of bacterial, fungal, protozoan or viral diseases, preferably bacterial and viral diseases. When the snake venom is from Bitis arietans or Trimersurus albolabris the above composition may be used preferably for viral diseases. The plant extract may in combination with either or a combination of the above snake venoms, form a substance which affects a virus in a following way: The viruses genetical influence on the invaded cell is weakened which will affect the cell so that it will not form virus replicates at the same pace as normal or will not form any replicates at all. The enzyme scissors of the viruses will not be able to operate fully or with the same force as on unaffected cells and will thus not cut off the molecules which are necessary for the virus to invade new cells.

According to yet another preferred embodiment of the present invention a pharmaceutical composition comprising at least two snake venoms from said two different groups of snakes (with neurotoxic venom and proteolytic venom, respectively; as above) and at least one plant extract, which preferably is obtainable from the plant family Ranunculaceae; and a pharmaceutically acceptable carrier is provided. The plant extract is preferably obtainable from the plant Aconitum septentrionale, Aconitum karacolicum or Aconitum napellus. Most preferred the plant extract is aconitine. Said composition may be used for the prevention, management or treatment of bacterial, fungal, protozoan or viral diseases, preferably bacterial and viral diseases, especially of special viral type e.g. HIV, Ebola, Herpes simplex or similar.

According to yet another preferred embodiment of the present invention a pharmaceutical composition adapted for oral administration is provided. This pharmaceutical composition adapted for oral administration comprises at least one snake venom or any other combination of venoms (or fractions or components thereof) and optionally a plant extract as is disclosed as preferred embodiments in the present specification; and a pharmaceutically acceptable carrier.

According to yet another preferred embodiment of the present invention snake venom, a fraction thereof or a component thereof obtainable from a snake comprised in the group Bungarus fasciatus, Ophiophagus hannah and Bungarus candidus, Lapemis hardwickil, Hydrophis cyanocinctus, Enhydrina schistosa, Aipysurus eydouxii, Bothrops atrox, Lachesis muta, Bitis arietans or Trimersurus albolabris for medical use is provided.

According to yet another preferred embodiment of the present invention, therapeutic use of a snake venom, a fraction thereof or a component thereof obtainable from a snake comprised in the group Bungarus fasciatus, Ophiophagus hannah and Bungarus candidus, Lapemis hardwickii, Hydrophis cyanocinctus, Enhydrina schistosa, Aipysurus eydouxii, Bothrops atrox, Lachesis muta, Bitis arietans or Trimersurus albolabris is provided.

According to yet another preferred embodiment of the present invention use of a snake venom, a fraction thereof or a component thereof obtainable from a snake comprised in the group Naja kaouthia, Bungarus fasciatus, Ophiophagus hannah and Bungarus candidus, Lapemis hardwickii, Hydrophis cyanocinctus, Enhydrina schistosa, Aipysurus eydouxii, Bothrops atrox, Lachesis muta, Bitis arietans, Trimersurus albolabris or aconitine in the manufacture of a medicament for the prevention, management or treatment of bacterial, fungal, protozoan or viral diseases is provided.

Pharmaceutical compositions according to the present invention contain a pharmaceutically acceptable carrier together with at least one of the snake venom as described herein, dissolved or dispersed therein as an active, antimicrobial, ingredient. In a preferred embodiment, the therapeutic composition is not immunogenic when administered to a human patient for therapeutic purposes, unless that purpose is to induce an immune response.

The preparation of a pharmacological composition that contains active ingredients dissolved or dispersed therein is well understood in the art. Typically such compositions are prepared as sterile injectables either as liquid solutions or suspensions, aqueous or non-aqueous, however, solid forms suitable for solution, or suspensions, in liquid prior to use can also be prepared. The preparation can also be emulsified.

The active ingredient may be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition may contain minor amounts of auxiliary substances such as wetting or emulsifying agents, pH buffering agents and the like which enhance the effectiveness of the active ingredient. Adjuvants may also be present in the composition. The venoms may themselves be used as adjuvants.

Pharmaceutically acceptable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, propylene glycol, polyethylene glycol and other solutes.

Liquid compositions can also contain liquid phases in addition to and to the exclusion of water. Exemplary of such additional liquid phases are glycerin, vegetable oils such as cottonseed oil, organic esters such as ethyl oleate, and water-oil emulsions.

The pharmaceutical composition according to one of the preferred embodiments of the present invention comprising at least one plant extract, preferably aconitine, may include pharmaceutically acceptable salts of that component therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

The preparations according to the preferred embodiments may be administered orally, topically, intraperitoneally, intraarticularly, intracranially, intradermally, intramuscularly, intraocularly, intrathecally, intravenously, subcutaneously. Other routes which are known for the skilled person in the art are thinkable.

The orally administrable compositions according to the present invention may be in the form of tablets, capsules, powders, granules, lozenges, liquid or gel preparations, such as oral, topical or sterile parenteral solutions or suspensions. Tablets and capsules for oral administration may be in unit dose presentation form and may contain conventional excipients such as binding agents, for example syrup, acacia, gelatin, sorbitol, traganath or polyvinyl-pyrrolidone; fillers e.g. lactose, sugar, maize-starch, calcium phosphate, sorbitol or glycine; tabletting lubricant e.g. magnesium stearate, talc, polyethylene glycol or silica; disintegrants e.g. potato starch, or acceptable wetting agents such as sodium lauryl sulphate. The tablets may be coated according to methods well known in normal pharmaceutical practice. Oral liquid preparations may be in the form of e.g. aqueous or oily suspensions, solutions, emulsions, syrups or elixirs or may be presented as a dry product for reconstitution with water or other suitable vehicle before use. Such liquid preparations may contain conventional additives such as suspending agents, e.g. sorbitol, syrup, methyl cellulose, glucose syrup, gelatin hydrogenated edible fats; emulsifying agents e.g. lecithin, sorbitan monooleate or acacia, non-aqueous vehicles (which may include edible oils), e.g. almond oil, fractionated coconut oil, oily esters such as glycerine, propylene glycol, or ethyl alcohol; preservatives e.g. methyl or propyl p-hydroxybenzoate or sorbic acid, and if desired conventional flavouring or colouring agents.

A pharmaceutical composition according to the present invention, comprises typically an amount of at least 0.1 weight percent of snake venom per weight of total therapeutic composition. A weight percent is a ratio by weight of snake venom to total composition. Thus, for example, 0.1 weight percent is 0.1 grams of snake venom per 100 grams of total composition. A suitable daily oral dose for a mammal, preferably a human being, may vary widely depending on the condition of the patient. However a dose of snake venom of about 0.1 to 300 mg/kg body weight may be appropriate.

The compositions according to the present invention may also be used veterinarily and thus they may comprise a veterinarily acceptable excipient or carrier.

According to yet another preferred embodiment of the present invention there is provided a method for purification of at least one pharmaceutically active fraction of a snake venom obtainable from a snake comprised in the group Ophiophagus hannah, Bitis arietans and Trimersurus albolabris, comprising the steps:

a) dissolving venom with one or more suitable agents to a final concentration of approximately 0.01 mM;

b) passing the dissolved venom diluted, approximately 1/10, through an ion exchange cationic column with approximately 1 ml capacity with a flow of approximately 0.5 ml/min; and

c) recovery of 40 fractions of approximately 600 μl each. Preferably the method involves the collection of the following fractions of step c):

for Ophiophagus hannah (OH): No. 1, 2, 3, 4, 5, 6, 7, 8, 12, 15, 16, 17, 18, 19, 20, 21, 22, 23, 24, 25, 26, 27, 28, 29, 30, 31, 32, 33, 34, 35, 36, 37, 38, 39, 40; most preferred No. 2, 3, 4, 15, 17, 18, 20, 23, 24, 30, 33, 34, 35 and 36; especially preferred No. 2, 3, 4, 15, 17, 18, 19, 20, 23, 24, 33, 34, 35, 36;

for Trimersurus albolabris (TA): No. 2, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 and 40; most preferred No. 13, 14, 15, 16, 17, 18, 19, 20, 21, 22, 23 and 24; especially preferred No. 13, 14, 15, 16, 17, 18, 19, 20, 21, 22, 23 and 24; and

for Bitis arietans (BA): No. 2, 3, 4, 5, 6, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 and 40; most preferred No. 3, 12, 13, 14, 15, 16, 17, 18, 19, 22, 23, 36 and 37; especially preferred No. 2, 3, 4, 11, 12, 13, 14, 15, 16, 17, 18, 19, 20, 21, 35, 36 and 37.

Preferably each venom is dissolved to a concentration of 0.2 mg/ul in 20 mM Ammonium Acetate pH 7, and PMSF (a protease inhibitor) is added to a final concentration of 0.01 mM and each vessel containing the diluted venoms is kept at 4° C. until used.

Preferably HiTrap® (Pharmacia Biotech) ion exchange cationic columns of 1 ml capacity is used for the separation of proteins according to the suppliers recommendations.

The column is preferably equilibrated with 0.05 M Ammonium Acetate and 1/10 dilution of the sample is applied to the column and eluted with continuous gradient from 0.05 to 1.4 M ammonium acetate (Ammon Ac).

The chromatography apparatus may preferably be a liquid chromatography controller LCC-500 (Pharmacia) run at 0.5 ml/min. At least fourty fractions of Six hundred μl each may be collected for each venom. They may preferably be put on ice until dried in a speedvaccum (Speedvac) centrifuge. Thus ammonium acetate may be removed. The ammonium acetate may itself have anti-bacterial effect, thus this removal eliminates a possible source of error by removing it.

The protein content may preferably be determined at 280 nm as the fractions flows out of the column. The dried pellets may preferably be resuspended in 200 μl 20mM NaCl and kept at 4° C.

According to yet another preferred embodiment of the present invention there is provided a fraction of a venom, preferably form OH, TA or BA, obtainable by the above method. The fraction may be used in a pharmaceutical composition as set out above.

According to yet another preferred embodiment of the present invention there is provided a pharmaceutical composition comprising a fraction of a venom obtainable by a method as set out above and a pharmaceutically acceptable carrier.

According to yet another preferred embodiment of the present invention there is provided a pharmaceutical composition comprising a component of a venom obtainable from Ophiophagus hannah, Bitis arietans or Trimersurus albolabris wherein the weight of the component is from approximately 20 to approximately 33 kDa; and a pharmaceutically acceptable carrier.

According to yet another preferred embodiment of the present invention there is provided a pharmaceutical composition comprising a component of a venom obtainable from Ophiophagus hannah wherein the weight of the component is approximately 20 or 33 kDa; and a pharmaceutically acceptable carrier.

According to yet another preferred embodiment of the present invention there is provided a pharmaceutical composition comprising two components of a venom obtainable from Ophiophagus hannah wherein the weights of the components are approximately 20 and 33 kDa, respectively; and a pharmaceutically acceptable carrier.

According to yet another preferred embodiment of the present invention there is provided a pharmaceutical composition comprising a component of a venom obtainable from Bitis arietans wherein the weight of the component is approximately 30 kDa; and a pharmaceutically acceptable carrier.

The invention will now be described in reference to the following figures and examples. These figures and examples are not to be regarded as limiting the scope of the present invention, but shall only serve in an illustrative manner.

### FIGURES

FIG. 1 shows the effect of venom from the snake Ophiophagus hannah (OH) on Staphylococcus epidermidis at different concentrations of OH.

FIG. 2 shows the effect of the combination of venoms Naja kaouthia, Bungarus fasciatus and Ophiophagus hannah (BON) on Salmonella typhimurium at different concentrations of BON.

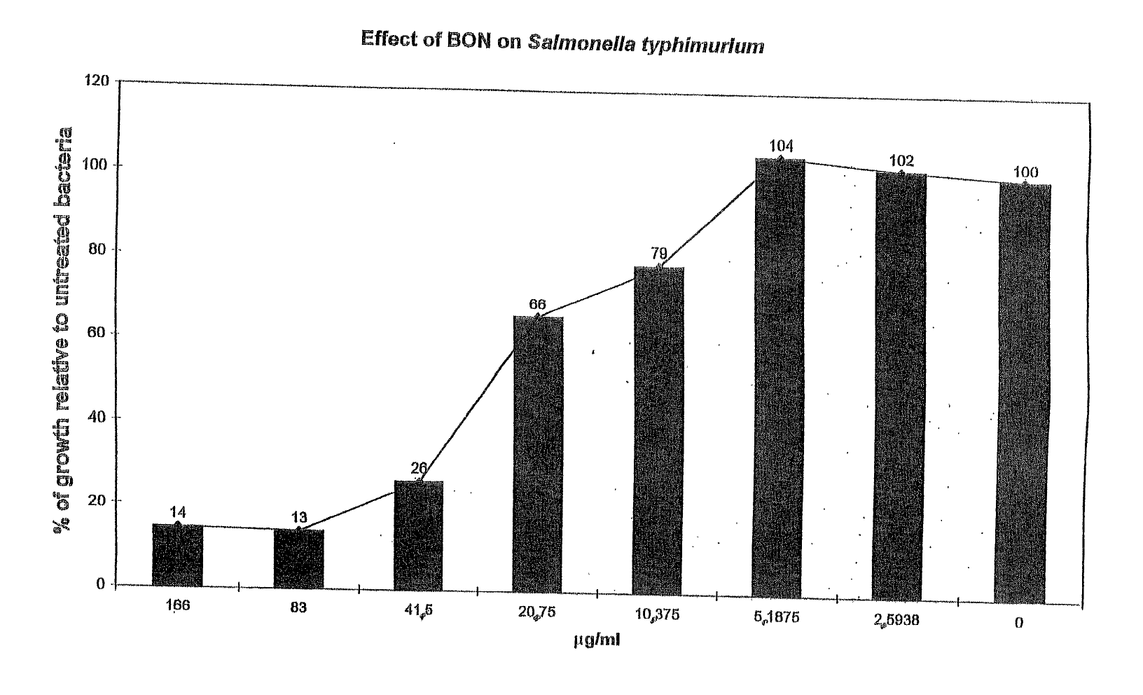

FIG. 3 shows the effect of single and combined venoms on Staphylococcus aureus at different venom concentrations.

FIG. 4 shows the effect of single and combined venoms on Salmonella typhimurium at different venom concentrations.

FIG. 5 shows the effect of single and combined venoms on Staphylococcus epidermis at different venom concentrations.

FIG. 6 shows the effect of single and combined venoms on Serratia marsescens at different venom concentrations.

FIG. 7 shows the effect of single and combined venoms on Streptococcus salivarius at different venom concentrations.

FIG. 8 shows the effect of single and combined venoms on Escherichia coli at different venom concentrations.

FIG. 9 shows the effect of single and combined venoms on Bacillus subtilis at different venom concentrations.

FIG. 10 shows the effect of single and combined venoms on Pseudomonas aeruginosa at different venom concentrations.

FIG. 11 shows the effect of single and combined venoms on Micrococcus intense at different venom concentrations.

FIG. 12 shows the effect of single and combined venoms on Mycobacterium smegmatis at different venom concentrations.

FIG. 13 shows the effect of single and combined venoms on Candida albicans at different venom concentrations.

FIG. 14 shows the effect of fractions, obtained by using a cationic ion-exchange column, of OH on Bacillus after 9 h.

FIG. 15 shows the effect of fractions of OH, obtained by using a cationic ion-exchange column, on E. coli after 9 h.

FIG. 16 shows the effect of fractions of OH, obtained by using a cationic ion-exchange column, on Bacillus after 21 h.

FIG. 17 shows the effect of fractions of OH, obtained by using a cationic ion-exchange column, on E. coli after 21 h.

FIG. 18 shows the effect of fractions of OH, obtained by using a cationic ion-exchange column, on Bacillus after 24 h.

FIG. 19 shows the effect of fractions of OH, obtained by using a cationic ion-exchange column, on E. coli after 24 h.

FIG. 20 shows the effect of fractions, obtained by using a cationic ion-exchange column, of TA on Bacillus after 9 h.

FIG. 21 shows the effect of fractions of TA, obtained by using a cationic ion-exchange column, on E. coli after 9 h.

FIG. 22 shows the effect of fractions of TA, obtained by using a cationic ion-exchange column, on Bacillus after 21 h.

FIG. 23 shows the effect of fractions of TA, obtained by using a cationic ion-exchange column, on E. coli after 21 h.

FIG. 24 shows the effect of fractions of TA, obtained by using a cationic ion-exchange column, on Bacillus after 24 h.

FIG. 25 shows the effect of fractions of TA, obtained by using a cationic ion-exchange column, on E. coli after 24 h.

FIG. 26 shows the effect of fractions , obtained by using a cationic ion-exchange column, of BA on Bacillus after 9 h.

FIG. 27 shows the effect of fractions of BA, obtained by using a cationic ion-exchange column, on E. coli after 9 h.

FIG. 28 shows the effect of fractions of BA, obtained by using a cationic ion-exchange column, on Bacillus after 21 h.

FIG. 29 shows the effect of fractions of BA, obtained by using a cationic ion-exchange column, on E. coli after 21 h.

FIG. 30 shows the effect of fractions of BA, obtained by using a cationic ion-exchange column, on Bacillus after 24 h.

FIG. 31 shows the effect of fractions of BA, obtained by using a cationic ion-exchange column, on E. coli after 24 h.

FIG. 32 shows the determination of protein content at 280 nm as the fractions flowed out of the column for the respective venom BA, OH and TA (designated FIG. 32(Ba), FIG. 32(Oh) and FIG. 32(Ta), respectively, in the figure). The gradient is also depicted (the straight line).

FIG. 33 shows the effect of fractions of OH, obtained by using a cationic ion-exchange column, on Bacillus subtilis after 12 h.

FIG. 34 shows the effect of fractions of OH, obtained by using a cationic ion-exchange column, on E. coli after 12 h.

FIG. 35 shows the effect of fractions of TA, obtained by using a cationic ion-exchange column, on E. coli.

FIG. 36 shows the effect of fractions of TA, obtained by using a cationic ion-exchange column, on Bacillus subtilis.

FIG. 37 shows the effect of fractions of BA, obtained by using a cationic ion-exchange column, on E. coli after 14 h.

FIG. 38 shows the effect of fractions of TA, obtained by using a cationic ion-exchange column, on Bacillus subtilis after 14 h.

FIG. 39 shows results of the toxicity tests.

FIG. 40 shows results of the toxicity tests.

FIG. 41 shows results of the toxicity tests.

FIG. 42 shows results of the toxicity tests.

FIG. 43 shows results of the toxicity tests.

FIG. 44 shows a gel picture showing the protein content in selected FPLC-fractions from OH after SDS-polyacrylamide gel electrophoresis (SDS-PAGE, 7.5% PA) and silver staining.

### EXPERIMENTAL

Generally for the experiments described in the following experimental part, the venoms from the Elapidae family were obtained from the Red Cross Snake Farm, Thailand and the Viperidae venoms were obtained from Venom Supplies Pty. Ltd, Tanuda, South Australia. Lyophilized venoms were reconstituted with Luria broth (LB) and YPD medium and sterilized by passage through a 0.22 μm filter.

The microbes that were used in the following experiments were kindly provided by Gunnel Dalhammar at the Department of Biochemistry and Biotechnology of the Royal Institute of Technology (KTH) in Stockholm, Sweden (bacterial strains: Bacillus subtilis, Staphylococcus aureus, Staphylococcus epidermidis, Streptococcus salivarius, Micrococcus intense, Escherichia coli K12, Salmonella typhimurium, Serratia marcescens and Pseudomonas aeruginosa), with the expections of Mycobacterium smegmatis (a mycobacterium) that was obtained from SMI (Smittskyddsinstitutet; SMI is the Swedish Institute for Infectious disease controll.) under the accession number ATCC 19420 and Candida albicans (a fungus) which was obtained under the ATCC number ATCC90028 from the central laboratory at the Karolinska Sjukhuset. The OD-monitoring device in all experiments was an E-max microplate precision reader from Molecular Devices Corp., California, USA. Reference antibiotics were Kanamycin (Gibco BRL), Penicillin—Streptomycin (also known as PEST) (Boehringer Mannheim), Chloramphenicol (Boehringer Mannheim) and Amphoterizine B (trademark Fungizone from Apoteket).The reference fungicide Amphoterizine B was in disolved form (manufacturer: Bristol Myers Squibb) and was obtained from the central laboratory at the Karolinska Sjukhuset (which in turn had obtained it from Apoteket).

Experiment 1

The first experiment, a screening, was performed by placing different bacteria at different concentrations in containers, microtiter plates, and then adding snake venoms or a reference antibiotic to the containers at different concentrations.

Venoms were: Naja kaouthia (NK), Bungarus fasciatus (BF) and Ophiophagus hannah (OH), all three combined: BON

Bacterias were: Staphylococcus epidermidis, Serratia marsescens, Escherichia coli K12, Salmonella typhimurium, Pseudomonas aeruginosa

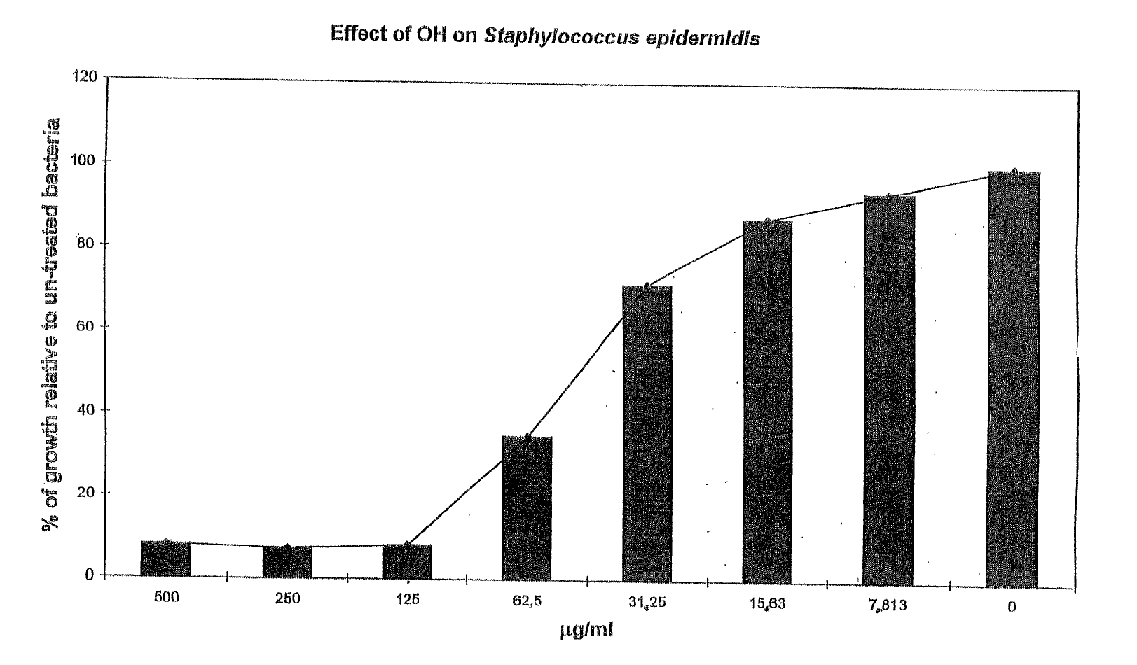

The concentrations for the bacteria were 3×10⁵-8×10⁵, 3×10⁴-8×10⁴ or 3×10³-8×10³ Colony Forming Units/ml (CFU/ml). The monitoring was performed by measuring optical density (OD) , CFU a.s.o. Dilutions and results (for BON, i.e. a mixture of BF, OH, and NK) can be seen in table 1. The results for OH is illustrated in FIG. 1.

TABLE 1
Dilutions of venom, antibiotic and results for BON

| | | | % growth |
|---|---|---|---|
| | | PEST or | relative to |
| BF, OH, NK | BON | Kanamycin | untreated |
| (μg/ml) | (μg/ml)* | (μg/ml) | bacteria (BON) |
| 500 | 166 | 20 | 14 |
| 250 | 83 | 10 | 13 |
| 125 | 41.5 | 5 | 26 |
| 62.5 | 20.75 | 2.5 | 66 |
| 31.2 | 10.375 | 1.2 | 79 |
| 15.6 | 5.1875 | 0.6 | 104 |
| 7.8 | 2.5938 | 0.3 | 102 |
| 0 | 0 | 0 | 100 |

*= Values are per venom
As can be seen in the results for BON in column 4 of table 1, there is an antibacterial effect above 5.1875 μg/ml concentration of BON. As can be seen in the results for OH, illustrated in FIG. 1, there is an antibacterial effect above 7.813 μg/ml concentration of OH.

Experiment 2

The second experiment consisted of a screening for antibiotic effect of individual and combined substances. The materials that were used were:

Venoms: from Elapidae, Naja kaouthia (NK), Bungarus fasciatus (BF) and Ophiophagus hannah (OH); from Viperidae, Bitis arietans (BA) and Trimersurus albolabris (TA)

Bacteria: Gram positive e.g. Bacillus subtilis, Streptococcus salivarius, Staphylococcus epidermidis, Micrococcus intense and Staphylococcus aureus; Gram negative e.g. Escherichia coli, P. aeruginosa, Serratia marsescens and Salmonella typhimurium;

Mycobacteria: Mycobacterium smegmatis.

The method involved the use of a microtiter (ELISA) plate (12×8 wells, i.e 96 wells in total, obtained from NUNC) with serial dilutions of venoms and bacteria at two concentrations.

Bacteria:

In columns 1-6 :×10⁵ bacteria/ml (Luria Broth(LB) medium)

In columns 7-12:×10⁴ bacteria/ml (LB medium)

Dilutions can be seen in table 2.

TABLE 2
Dilutions of venom and antibiotic

| Column of | Venom dilutions | Antibiotic dilution |
|---|---|---|
| ELISA plate | (μg/ml) | (μg/ml) (reference) |
| 1 | 500 | 100 |
| 2 | 250 | 50 |
| 3 | 125 | 25 |
| 4 | 62.5 | 12.5 |
| 5 | 31.2 | 6.25 |
| 6 | 0 | 0 |
| 7 | 500 | 100 |
| 8 | 250 | 50 |
| 9 | 125 | 25 |
| 10 | 62.5 | 12.5 |
| 11 | 31.2 | 6.25 |
| 12 | 0 | 0 |

The incubation time was 24 hours. OD was monitored at 650 nm every hour the first 7-8 hours, and then at 20 hours and 24 hours post inoculation. Each well was given 200 μl of diluted venom plus bacteria.

The results were as follows:

For E. coli: (7 hours post inoculation)

Antibacterial effect of all single venoms. The effect of Ophiophagus hannah, Bitis arietans and Trimersurus albolabris, respectively, was in degree with reference antibiotic Kanamycin. The effect of Naja kaouthia and Bungarus fasciatus was weaker for conditions of lower venom concentrations. No synergistic effect from combining Ophiophagus hannah and any of the Viperidae venom.

For E. coli: (24 hours post inoculation)

Weaker antibacterial effect of single venoms compared to 7 hours post inoculation, except for at high concentration of Ophiophagus hannah and Trimersurus albolabris. Certain synergistic effect from combining Ophiophagus hannah and any of the Viperidae venoms could be seen.

For S. aureus: (7 hours post infection)

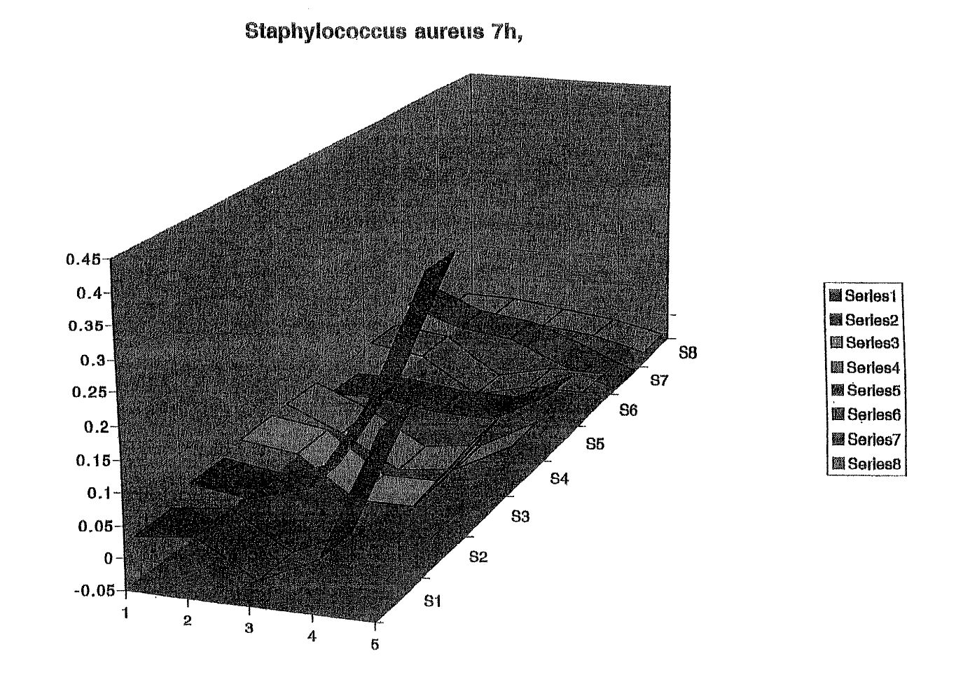

Antibacterial effect for all single venoms. FIG. 3 shows that OH and BA has a strong effect on the growth. Also OH combined with BA and OH combined with TA show a strong antibacterial effect.

For S. aureus: (20 hours post infection)

Antibacterial effect of all single venoms. The effect of Ophiophagus hannah, Bitis arietans and Trimersurus albolabris, respectively, was in degree with reference antibiotic PEST (i.e. Penicillin and Streptomycin), especially Ophiophagus hannah. The effect of Naja kaouthia and Bungarus fasciatus was weaker for conditions of lower venom concentrations. No synergistic effect detectable from combining Ophiophagus hannah and any of the Viperidae venoms.

For B. subtilis: (20 hours post infection)

Antibacterial effect of all single venoms. The effect Ophiophagus hannah, Bitis arietans and Trimersurus albolabris, respectively, was stronger when compared with reference antibiotic PEST, especially Ophiophagus hannah. The effect of Naja kaouthia and Bungarus fasciatus was weaker for conditions of lower venom concentrations. The effect of Bungarus fasciatus was even weaker. A synergistic effect from combining Ophiophagus hannah and any of the Viperidae venoms was in this case however found.

For Salmonella typhimurium: (7 hours post infection)

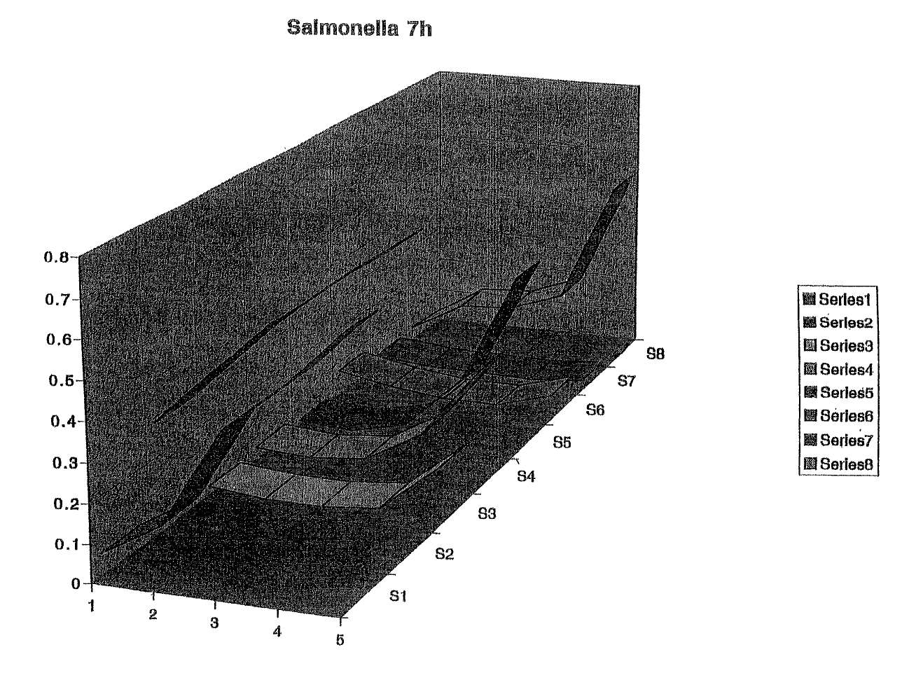

Antibacterial effect was seen for all single venoms, however somewhat weaker for NK. FIG. 4 shows that OH and BA has an effect on the growth. OH combined with BA and OH combined with TA show a strong antibacterial effect.

For Staphylococcus epidermidis: (7 hours post infection)

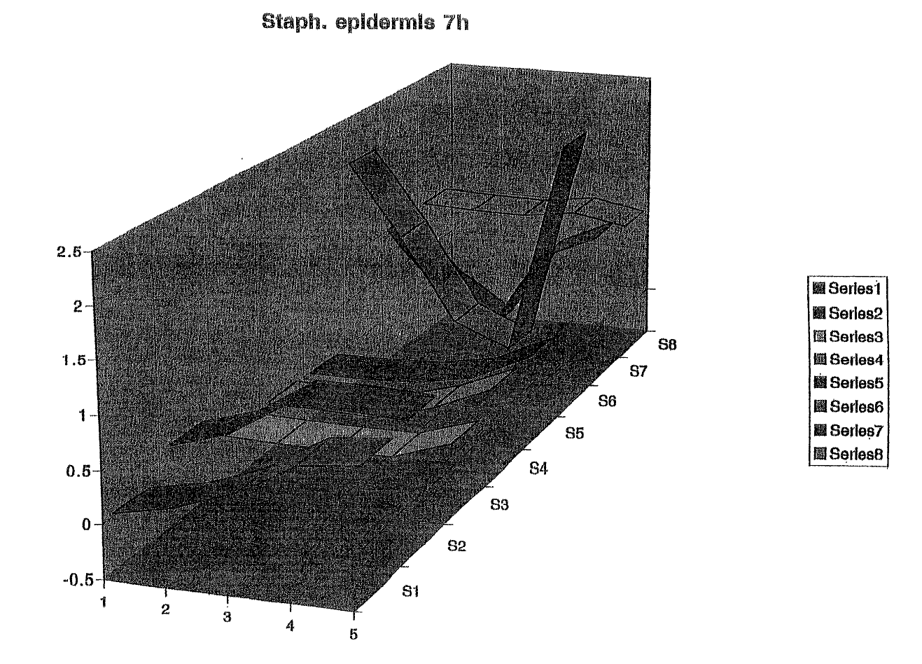

Antibacterial effect was seen for all single venoms. FIG. 5 shows that OH, BA and TA have a strong effect on the growth. OH combined with BA and OH combined with TA show partially a strong antibacterial effect.

For Serratia marsescens: (7 hours post infection)

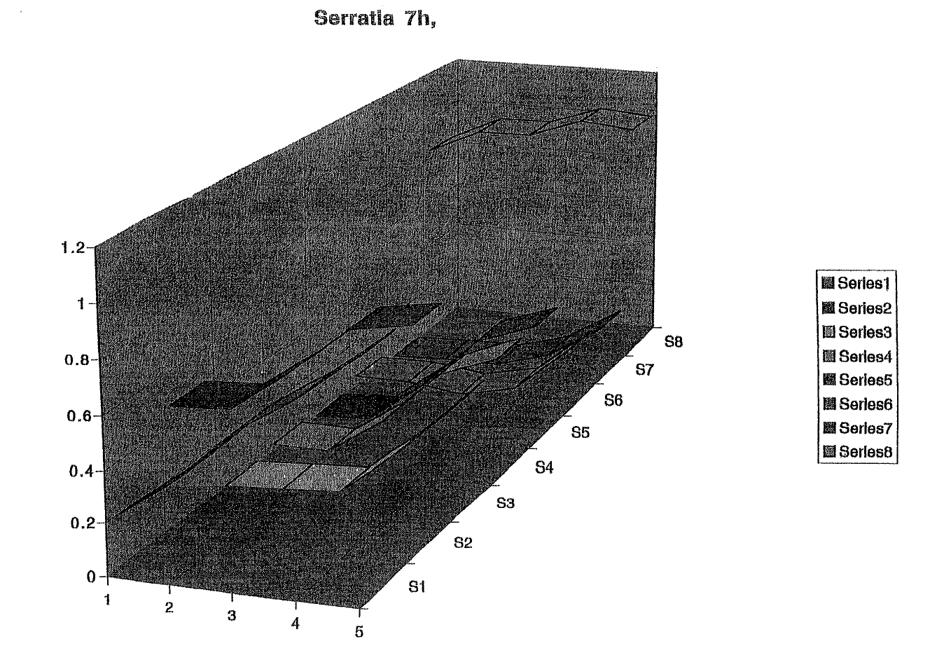

Antibacterial effect was seen for all single venoms. FIG. 6 shows that OH, BA and TA have a strong effect on the growth. OH combined with BA and OH combined with TA show an even stronger antibacterial effect.

For Streptococcus salivarius: (54 hours post infection)

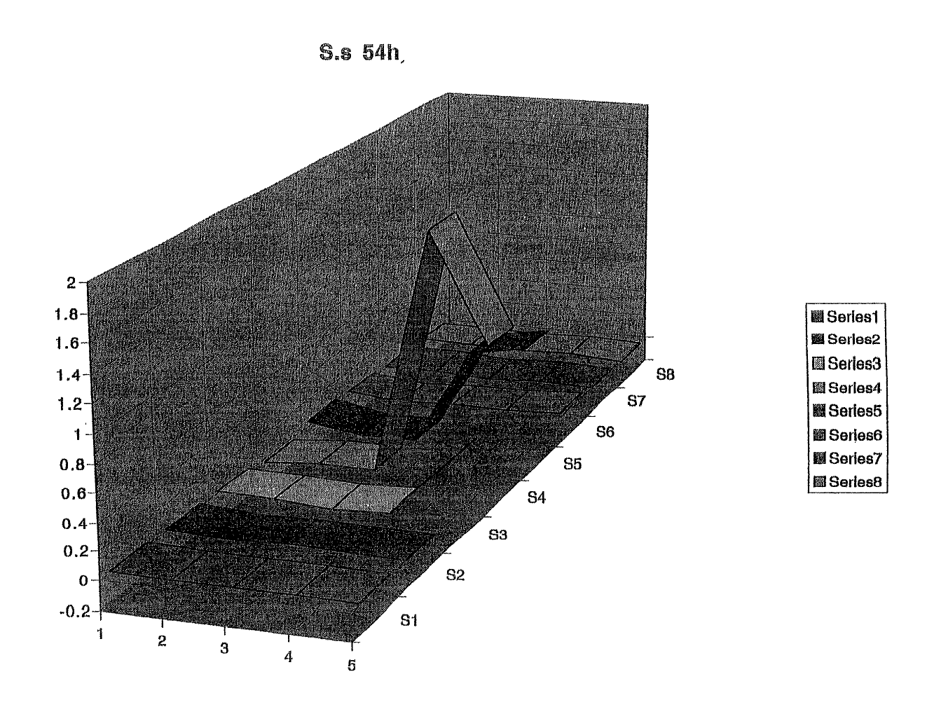

Antibacterial effect was seen for all single venoms. FIG. 7 shows that BA and TA have partially a somewhat weaker effect, whereas the other, and combinations, were more efficient.

For P. aeruginosa: (7 h post inoculation)

Antibacterial effect of all single venoms. The reference antibiotic Kanamycin had almost no effect. The effect of OH, BA and TA was very good. Combined venoms had a similar effect as OH

For P. aeruginosa: (24 h post inoculation)

Weaker antibacterial effect of single venoms compared to 7 h post inoculation. OH effect was still strong at high concentrations. The reference antibiotic Kanamycin had no effect. Combined venoms had the similar effect as OH.

For Micrococcus intense: (7 h post inoculation)

Antibacterial effect of all single venoms, being stronger than the reference antibiotic PEST (comparing concentration 1:1), which had almost no effect. OH, BA and TA had very strong effect. Combined venoms also had antibacterial effect.

For Micrococcus intense: (24 h post inoculation)

Weaker antibacterial effect of single venoms compared to 7 h post inoculation. OH effect was still strong at high concentrations. The reference antibiotic PEST had no effect. Combined venoms had the similar effect as OH.

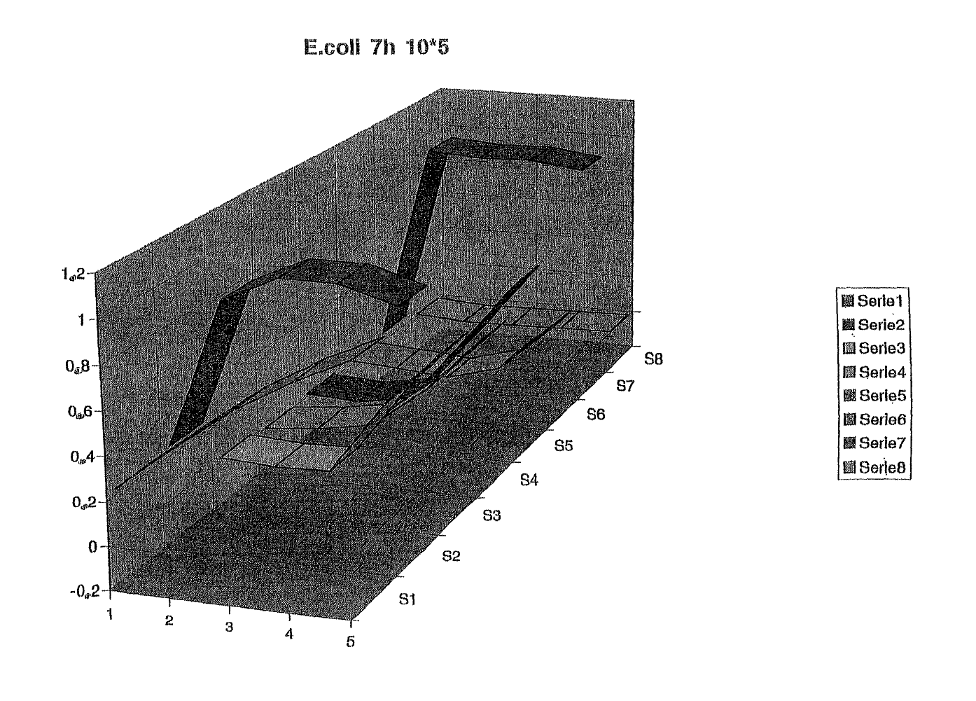

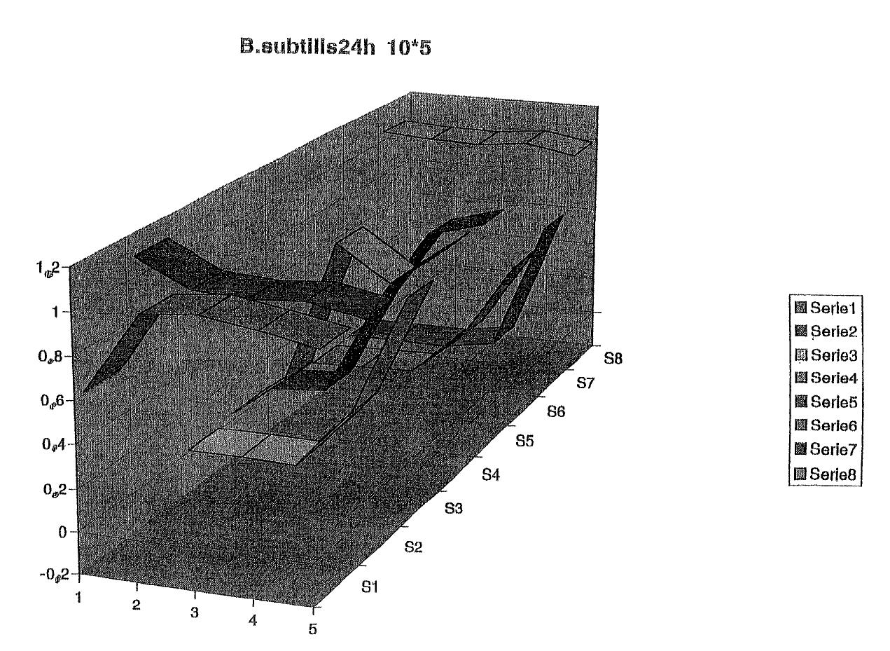

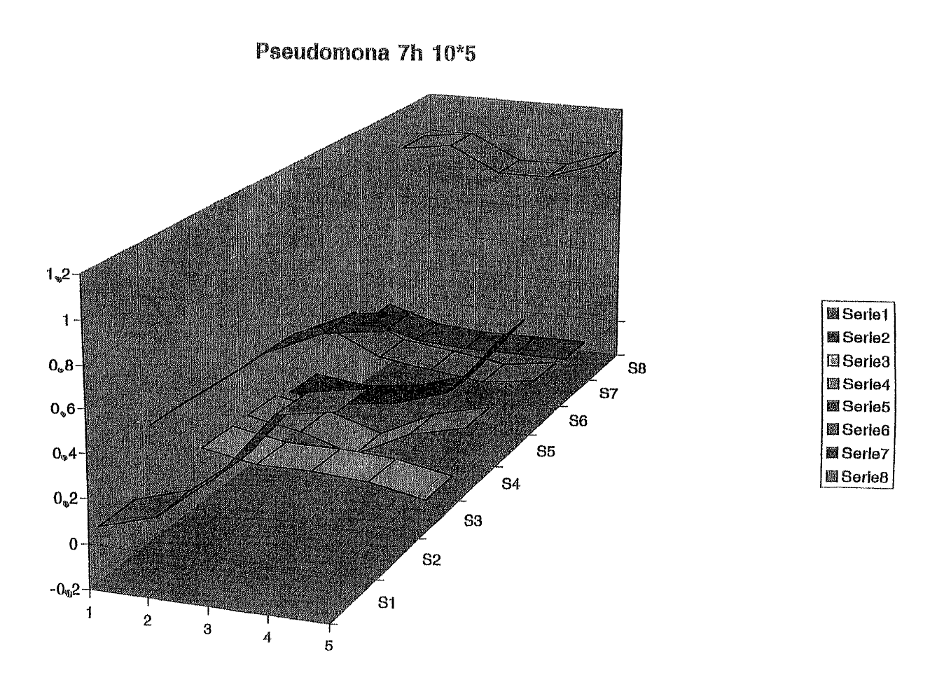

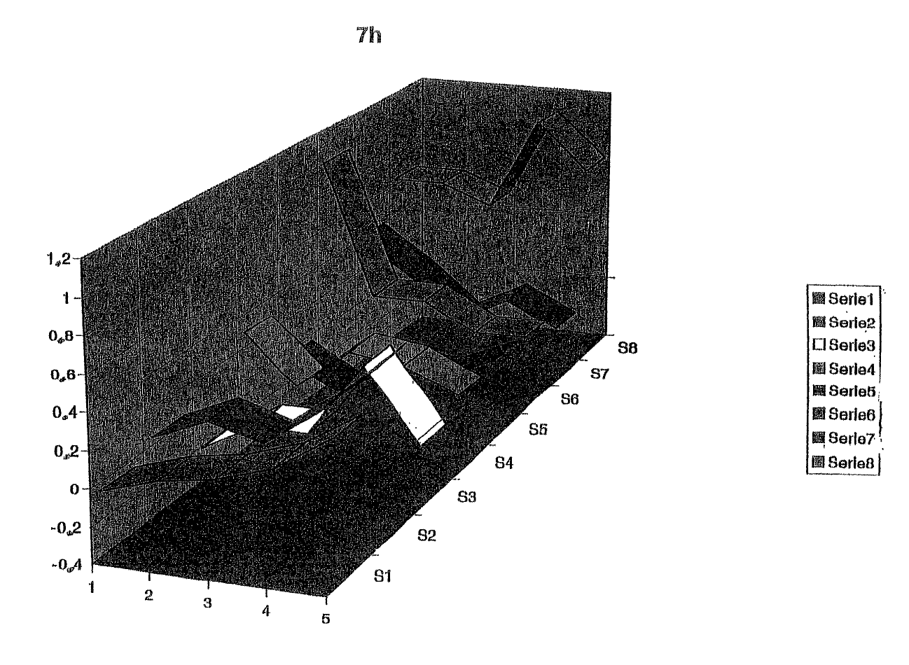

Three-dimensional diagrams shown as FIG. 3 to 11 summarize the above results. Observe that in each diagram serge 1 (Z axis) is substance BF, serie 2 is NK, 3 is OH, 4 is BA, 5 is TA, 6 is OH+BA, 7 is OH+TA and serie 8 is reference antibiotic. On the X-axis 1-5 is concentration of substance: 500, 250, 125, 62.5 och 31.2 mikrog/ml for substances (N.B. OH+BA and OH+TA have the double concentrations, i.e. the concentration is 1000, 500, 250, 125 and 62.5 ), and 100, 50, 25, 12.5, 6.25 mikrog/ml for reference antibiotic. The name on the diagrams is the tested bacteria strain (initials only given) and time is 7 hours after incubation start at which the substance was added. The combined OH+BA and OH+TA were obtained by mixing the single venoms in the same proportions.

Experiment 3

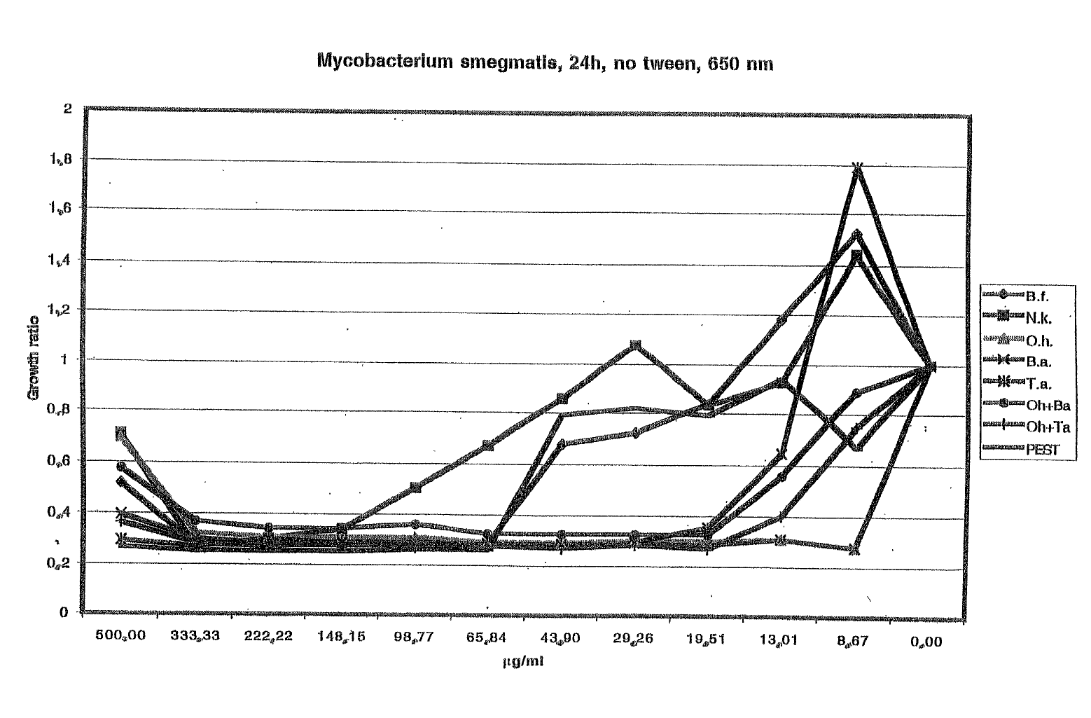

This experiment was a similar procedure as Experiment 2, but the test microbe was the bacteria Mycobacterium smegmatis (which is closely realted to tuberculosis and lepra bacilli). Venoms from Ophiophagus hannah and Bitis arietans among others, were used. An effect for all the venoms individually and combined was seen. FIG. 12, which refers to this third experiment, shows the results from which you can see that OH and BA has a very strong effect on the growth ratio for M. smegmatis, i.e. OH and BA has individually a strong antibacterial effect. The incubation time was 24 hours. Also OH combined with BA and OH combined with TA show a strong antibacterial effect. BF, NK, OH, BA, TA, OH+BA, OH+TA and reference antibiotic PEST were used. OD was monitored at 650 nm. Tween which usually is used for separating the cells was omitted in the experiment. The growth medium was DUBOS from Difco. Further experiments have shown that all venoms described above in Experiments 1 to 3, have a bactericidal effect.

Experiment 4

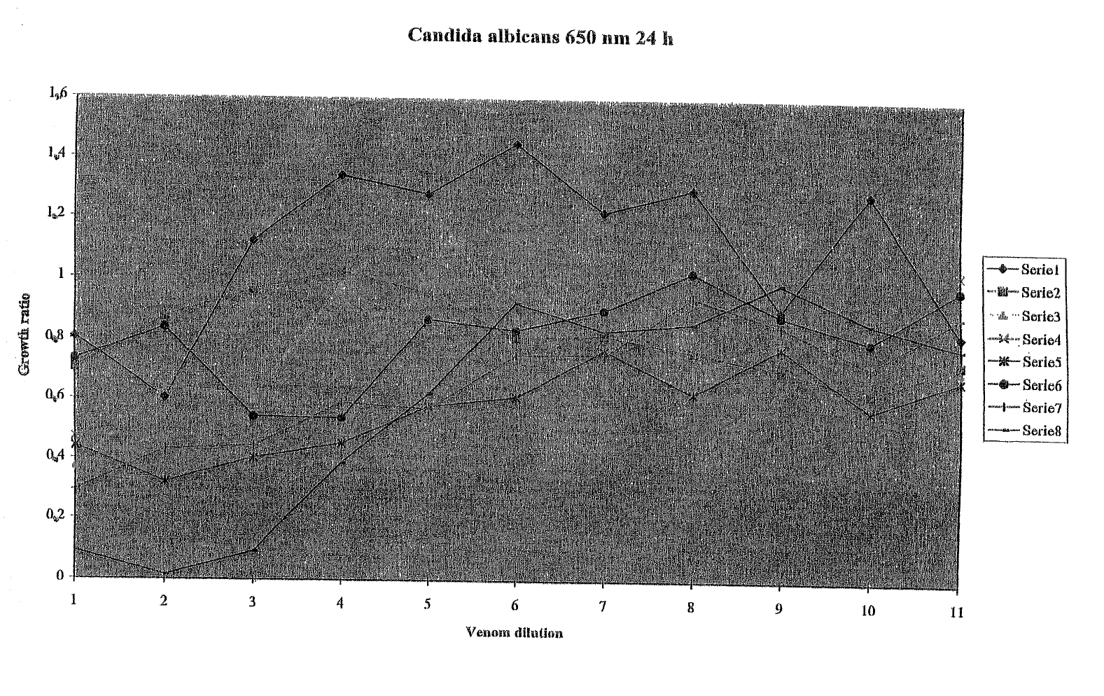

This experiment was a similar procedure as Experiment 2, but the test microbe was Candida albicans (a fungus) and only one cell concentration was used on the whole plate, i.e. 10⁵ cells/ml. Candida albicans is a yeast fungus which may cause problems on humans. The dilutions were for well 1-11, with start in well 1: 500 μg/ml as earlier and halving of the concentration at each subsequent step until finally reaching well 11. The reference fungicide was Amphoterizine B at dilutions starting at 2.5 μg/ml and then serial dilutions in 11 steps as for the bactericides. The series are from 1 to 8: BF, NK, OH, BA, TA, OH+BA, OH+TA and reference fungicide, respectively. The results are illustrated in FIG. 13. As can be seen there was an antifungal effect of OH, BA and TA. Combined venoms had similar effect as single venoms.

Experiment 5: Susceptibility studies

The capacity of the various venoms and combinations of them to inhibit microbial growth was measured as minimal inhibitory concentration (MIC). The capacity of the venoms to kill microbes was measured as minimal bacteriocidal concentration (MBC). Briefly, log phase bacteria and mycobacteria were cultured at densities 10⁵ and 10³ cells / ml in the presence of serial dilutions of each venom in LB medium starting at 500 μg venom / ml in a total volume of 200 μl/well in microtiter plates. After 7 and 24 h of incubation at 37° C., the growth of the microbes was measured by means of optical density (OD) at 650 and 405 nm using an E-max microtiter plate precision reader (Molecular Devices Corp., California, USA). For slow growers the measurement was done after longer incubations, (see Tables 3-8). MIC was defined as the venom concentration needed for no measurable microbe growth. Immediately after OD measurement for-MIC, 5 μl of microbe culture from each microtiter plate well was transferred to a corresponding well containing 195 μl LB medium in a new microtiter plate. The new plate was incubated at 37° C. for 24 h and subsequenctly subjected to OD measurements. MBC was defined as the venom concentration at which no growth was observed in this plate. The venom activity on Candida was determined similarity, but the culture medium was YPD. The antimicrobial activity of single venoms as well as of combined venoms was determined. The experiments with inoculum size of 10⁵ bacteria per ml were performed two or three times.

Results

The three venoms from O. hannah, B. arietans and T. albolabris inhibit growth of all the microbes tested within the venom concentrations range tested. The venom from O. hannah has a strong effect. The same three venoms, at the concentration of 500 μg/ml, are able to kill two of the bacterial strains ( See Tables 3-8). The venom from B. fasciatus displayed a weaker antimicrobial effect compared to the other venoms. Combination of the venoms from O. hannah and T. albolabris had an enhanced antibacterial effect on Streptococcus salivarius and Micrococcus intense, above additive level. A similar additive effect was seen when treating M. intense with a combination of the venoms from O. hannah and B. arietans.

TABLE 3
Antimicrobial effect of Ophiophagus hannah, inoculum
10⁵ microbe cells per ml (1-3 experiments per microbe were
performed).

| | MIC | OD | MIC | OD | Rela- | | OD | |
|---|---|---|---|---|---|---|---|---|
| | 7h | 7h | 24h | 24h | tive | MBC | con- | Relative |
| | mg/ | con- | (mg/ | con- | growth | (mg/ | trol | growth |
| Microbe | ml) | trol | ml) | trol | 24h | ml) | MBC | MBC |
| Bacillus | | 0.00 | 125 | 1.0 | 0% | ND | | |
| subtilis | ND | | >500 | 1.1 | 38% | >500 | 1.0 | 69% |
| Staphylo- | 31.2 | 0.1 | 62.5 | 0.3 | 0% | ND | | |
| coccus | ND | | 250 | 0.5 | 0% | 500 | 0.4 | 0% |
| aureus | | | | | | | | |
| Staphylo- | 62.5 | 0.2 | 500 | 1.4 | 0% | ND | | |
| coccus | ND | | >500 | 1.5 | 57% | >500 | 1.3 | 100% |
| epidermidis | | | | | | | | |
| Streptococcus | | 0.00 | 62.5 | 0.2* | 0% | ND | | |
| salivarius | | | 125 | 0.2 | 0% | 500 | 0.3* | 0% |
| Micrococcus | | 0.00 | 125 | 1.0 | 0% | ND | | |
| intense | ND | | >500 | 1.0 | 80% | >500 | 0.8 | 74% |
| Escherichia | 125 | 0.2 | 500 | 0.75 | 0% | ND | | |
| coli | | | | | | | | |
| K12 | ND | | 500 | 0.75 | 0% | ND | | |
| | ND | | >500 | 1.1 | 57% | >500 | 0.7 | 100% |
| Salmonella | 62.5 | 0.2 | 500 | 1.0 | 0% | ND | | |
| typhimurium | ND | | >500 | 0.8 | 45% | >500 | 0.6 | 100% |
| Serratia | 125 | 0.3 | >500 | 1.3 | 60% | ND | | |
| marcescens | ND | | >500 | 1.1 | 23% | >500 | 0.8 | 72% |
| Pseudomonas | 31.2 | 0.1 | >500 | 0.9 | 49% | ND | | |
| aeruginosa | ND | | >500 | 1.1 | 3% | >500 | 0.7 | 89% |
| Mycobacterium | ND | | 44 | 0.4\hat{ } | 0% | ND | | |
| smegmatis | | | | | | | | |
| Candida | | 0.00 | >500 | 0.4 | 42% | ND | | |
| albicans | | | | | | | | |

MIC 7h: minimal inhibitory concentration after 7 h incubation
MBC: minimal inhibitory concentration
OD 7h control: optical density of microbe culture without any venom after 7 h incubation
Relative growth 24h: optical density of microbe culture with venom at the venom concentration given after 24h incubation, divided by OD 24 h control.
ND: not determined
*: after 51 h incubation
^{\hat{ }}: after 120 h incubation

TABLE 4
Antimicrobial effect of Ophiophagus hannah, inoculum
Microbe

| | MIC | | | | | |
|---|---|---|---|---|---|---|
| | 24h | OD | Relative | MBC | OD | relative |
| | (mg/ | con- | growth | (mg/ | con- | growth |
| Microbe | ml) | trol | MIC | ml) | trol | MBC |
| Bacillus subtilis | 500 | 1.0 | 0% | >500 | 1.0 | 100% |
| Staphylococcus | 250 | 0.9 | 0% | 500 | 1.0 | 0% |
| epidermidis | | | | | | |
| Micrococcus intense | 500 | 1.2 | 0% | >500 | 1.0 | 72% |
| Escherichia coli K12 | 500 | 0.8 | 0% | >500 | 0.8 | 100% |
| Salmonella | 500 | 0.8 | 0% | >500 | 0.8 | 72% |
| typhimurium | | | | | | |
| Serratia marcescens | 500 | 1.3 | 0% | >500 | 1.0 | 100% |
| Pseudomonas | >500 | 0.9 | 5% | >500 | 0.9 | 76% |
| aeruginosa | | | | | | |
| Mycobacterium | | | | | | |
| smegmatis | | | | | | |
| Candida albicans | >500 | 0.4 | 22% | >500 | 0.5 | 34% |

TABLE 5
Antimicrobial effect of Bitis arietans, inoculum 10⁵ microbe
cells per ml (1-3 experiments per microbe were performed).

| | MIC | OD | MIC | OD | Relative | | OD | Relative |
|---|---|---|---|---|---|---|---|---|
| | 7 h | 7 h | 24 h | 24 h | growth | MBC | control | growth |
| Microbe | (mg/ml) | control | (mg/ml) | control | 24 h | (mg/ml) | MBC | MBC |
| Bacillus | | 0.00 | >500 | 1.0 | 3% | ND | | |
| subtilis | | | >500 | 1.1 | 80% | >500 | 0.9 | 100% |
| Staphylococcus | 31.2 | 0.1 | 62.5 | 0.3 | 0% | ND | | |
| aureus | | | 250 | 0.5 | 0% | 500 | 0.4 | 0% |
| Staphylococcus | 125 | 0.2 | >500 | 1.1 | 31% | ND | | |
| epidermidis | | | >500 | 1.5 | 100% | >500 | 1.2 | 100% |
| Streptococcus | | 0.00 | 125 | 0.2* | 0% | ND | | |
| salivarius | | | 250 | 0.3 | 0% | 250 | 0.4 | 0% |
| Micrococcus | | 0.00 | 500 | 1.0 | 0% | ND | | |
| intense | | | >500 | 0.9 | 100% | >500 | 0.7 | 80% |
| Escherichia | 125 | 0.15 | >500 | 0.8 | 41% | ND | | |
| coli K12 | | | >500 | 0.7 | 43% | ND | | |
| | | | >500 | 1.1 | 82% | >500 | 0.7 | 100% |
| Salmonella | 125 | 0.2 | >500 | 1.0 | 56% | ND | | |
| typhimurium | | | >500 | 0.8 | 100% | >500 | 0.6 | 100% |
| Serratia | 250 | 0.3 | >500 | 0.3 | 94% | ND | | |
| marcescens | | | >500 | 1.1 | 100% | >500 | 0.9 | 100% |
| Pseudomonas | 31.2 | 0.1 | >500 | 0.9 | 100% | ND | | |
| aeruginosa | | | >500 | 1.1 | 100% | >500 | 0.7 | 100% |
| Mycobacterium | ND | | 99 | 0.4ˆ | | ND | | |
| smegmatis | | | | | | | | |
| Candida | | 0.00 | >500 | 0.4 | 42% | ND | | |
| albicans | | | | | | | | |

TABLE 6
Antimicrobial effect of Bitis arietans, inoculum 10³
microbe cells per ml

| | MIC | OD | Relative | MBC | OD | Relative |
|---|---|---|---|---|---|---|
| | 24h | con- | growth | (mg | con- | growth |
| Microbe | ml) | trol | MIC | ml) | trol | MBC |
| Bacillus subtilis | >500 | 1.0 | 100% | >500 | 1.0 | 100% |
| Staphylococcus | >500 | 0.9 | 41% | >500 | 0.9 | 100% |
| epidermidis | | | | | | |
| Micrococcus intense | >500 | 1.2 | 100% | >500 | 1.2 | 100% |
| Escherichia coli K12 | >500 | 0.8 | 100% | >500 | 0.8 | 100% |
| Salmonella | >500 | 0.8 | 75% | >500 | 0.8 | 100% |
| typhimurium | | | | | | |
| Serratia marcescens | >500 | 1.3 | 79% | >500 | 1.0 | 100% |
| Pseudomonas | >500 | 0.9 | 61% | >500 | 0.9 | 100% |
| aeruginosa | | | | | | |
| Mycobacterium | | | | | | |
| smegmatis | | | | | | |
| Candida albicans | >500 | 0.4 | 100% | >500 | 0.5 | 100% |

TABLE 7
Antimicrobial effect of Trimersurus albolabris, inoculum 10⁵
microbe cells per ml (1-3 experiments per microbe were performed).

| | MIC | OD | MIC | OD | Relative | | OD | Relative |
|---|---|---|---|---|---|---|---|---|
| | 7 h | 7 h | 24 h | 24 h | growth | MBC | control | growth |
| Microbe | (mg/ml) | control | (mg/ml) | control | 24 h | (mg/ml) | MBC | MBC |
| Bacillus | | 0.00 | 250 | 1.0 | 0% | ND | | |
| subtilis | | | >500 | 1.1 | 73% | >500 | 0.9 | 100% |
| Staphylococcus | 31.2 | 0.1 | 125 | 0.3 | 0% | ND | | |
| aureus | | | 250 | 0.5 | 0% | 250 | 0.4 | 0% |
| Staphylococcus | 125 | 0.2 | >500 | 1.1 | 28% | ND | | |
| epidermidis | | | >500 | 1.5 | 100% | >500 | 1.2 | 100% |
| Streptococcus | | 0.00 | 125 | 0.2* | 0% | ND | | |
| salivarius | | | 250 | 0.3 | 0% | 250 | 0.4 | 0% |
| Micrococcus | | 0.00 | 500 | 1.0 | 0% | ND | | |
| intense | | | >500 | 0.9 | 100% | >500 | 0.7 | 90% |
| Escherichia | 125 | 0.15 | >500 | 0.75 | 28% | ND | | |
| coli K12 | | | >500 | 0.7 | 28% | ND | | |
| | | | >500 | 1.1 | 91% | >500 | 0.7 | 100% |
| Salmonella | 125 | 0.2 | >500 | 1.0 | 40% | ND | | |
| typhimurium | | | >500 | 0.8 | 100% | >500 | 0.6 | 100% |
| Serratia | 250 | 0.3 | >500 | 0.3 | 79% | ND | | |
| marcescens | | | >500 | 1.1 | 100% | >500 | 0.9 | 100% |
| Pseudomonas | 62.5 | 0.1 | >500 | 0.9 | 68% | ND | | |
| aeruginosa | | | >500 | 1.1 | 100% | >500 | 0.7 | 100% |
| Mycobacterium | | ND | 99 | 0.4ˆ | 0% | ND | | |
| smegmatis | | | | | | | | |
| Candida | | 0.00 | >500 | 0.4 | 50% | ND | | |
| albicans | | | | | | | | |

TABLE 8
Antimicrobial effect of Trimersurus albolabris,
inoculum 10³ microbe cells per ml

| | MIC | | | | | |
|---|---|---|---|---|---|---|
| | 24h | OD | Relative | MBC | OD | Relative |
| | (mg/ | con- | growth | (mg/ | con- | growth |
| Microbe | ml) | trol | MIC | ml) | trol | MBC |
| Bacillus subtilis | >500 | 1.0 | 90% | >500 | 1.0 | 100% |
| Staphylococcus | >500 | 0.9 | 7% | >500 | 0.9 | 100% |
| epidermidis | | | | | | |
| Micrococcus intense | >500 | 1.2 | 100% | >500 | 1.2 | 100% |
| Escherichia coli K12 | >500 | 0.8 | 68% | >500 | 0.8 | 100% |
| Salmonella | >500 | 0.8 | 41% | >500 | 0.8 | 100% |
| typhimurium | | | | | | |
| Serratia marcescens | >500 | 1.3 | 74% | >500 | 1.0 | 100% |
| Pseudomonas | >500 | 0.9 | 60% | >500 | 0.9 | 100% |
| aeruginosa | | | | | | |
| Mycobacterium | | | | | | |
| smegmatis | | | | | | |
| Candida albicans | >500 | 0.4 | 22% | >500 | 0.5 | 22% |

Experiment 6

Fractionation of OH, BA and TA through ion exchange.

The substances referred to as OH, BA and TA above, are snake venoms that contain a mixture of components. It was found in previous experiments above that such venoms displayed antibiotic activities against several microorganisms. As a first step to identifying the antibiotic substances (components) contained in the venoms a fractionation procedure was applied. Initially the venoms were characterized in terms of their subcomponents according to molecular weight and pH. The separation of venom components of peptidic origin according to molecular weight was performed through Sodium Dodecyl-Sulfate Poly-Acrylamide Gel Electrophoresis (SDS-PAGE). The protein or peptide subcomponents of each venom migrated in gels producing a pattern of bands that was compared to a standard molecular weight marker in order to determine the range of molecular weights of proteins or peptides in each venom.

Each venom was also electrophoresed through agarose gels that had gradients of pH values. After migration through such gels it was possible to determine the pattern of bands produced under acidic or basic pH. This information was used to determine the conditions needed for the separation of venoms at a preparative level.

Preparative separation of venom subcomponents with FPLC:

1. Fractionation (no protease inhibitors)

a. Each venom was dissolved to a concentration of 0.2 mg/ul in 20 mM Ammonium Acetate pH 7, PMSF (a protease inhibitor) was added to a final concentration of 0.01 mM and each vial was then kept at 4° C. until used. The stock solution of the venoms was prepared at 200 ug/ul.

b. HiTrap® (Pharmacia Biotech) ion exchange cationic columns of 1 ml capacity were used for the separation of proteins according to the suppliers recommendations.

c. The column was equilibrated with 0.05 M Ammonium Acetate and 1/10 dilution of the sample was applied to the column and eluted with a continuous gradient from 0.05 to 1.4 M Ammonium Acetate (Ammon Ac). The solution comprising venom thus was diluted 1:10. Two hundred microliters of the diluted venom were loaded on the column. Therefore 200 ul loaded×20 ug/ul=4 mg loaded on the column. The solvent used for dilution was 20 mM Ammonium Acetate

d. The chromatography apparatus used was a liquid chromatography controller LCC-500 (Pharmacia) run at 0.5 ml/min.

e. Fourty fractions of Six hundred μl each were collected for each venom. They were put on ice until dried in a speedvaccum centrifuge.

f. The protein content was determined at 280 nm as the fractions flowed out of the column.

g. The dried pellets were resuspended in 200 μl 20 mM NaCl and kept at 4° C.

2. Antibiotic activity testing

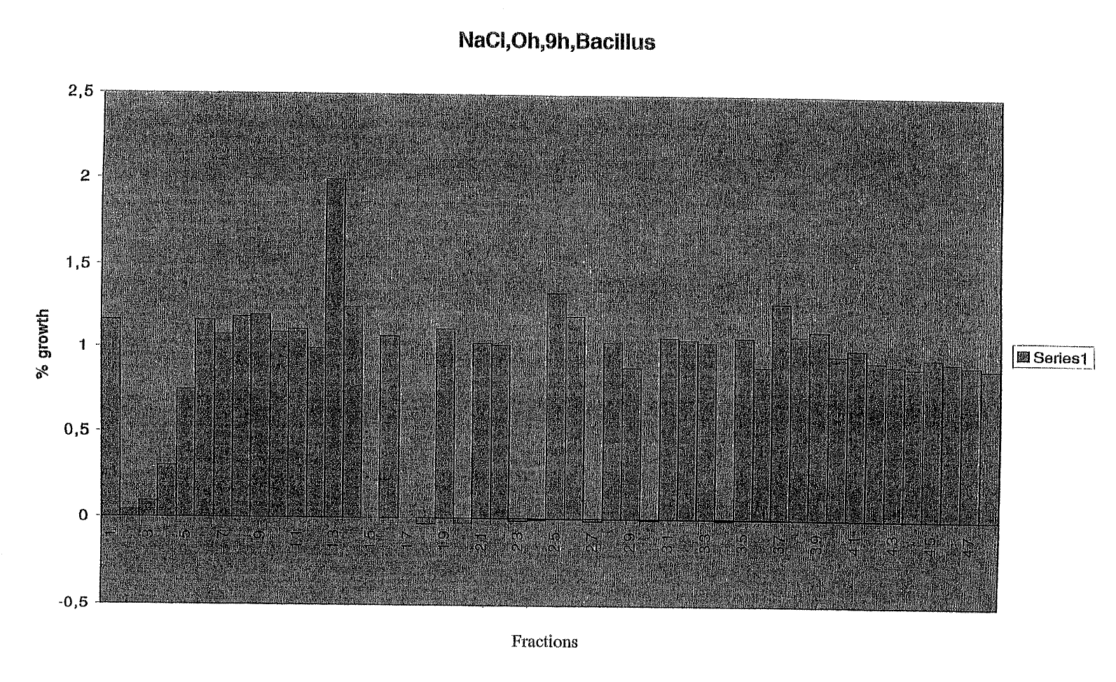

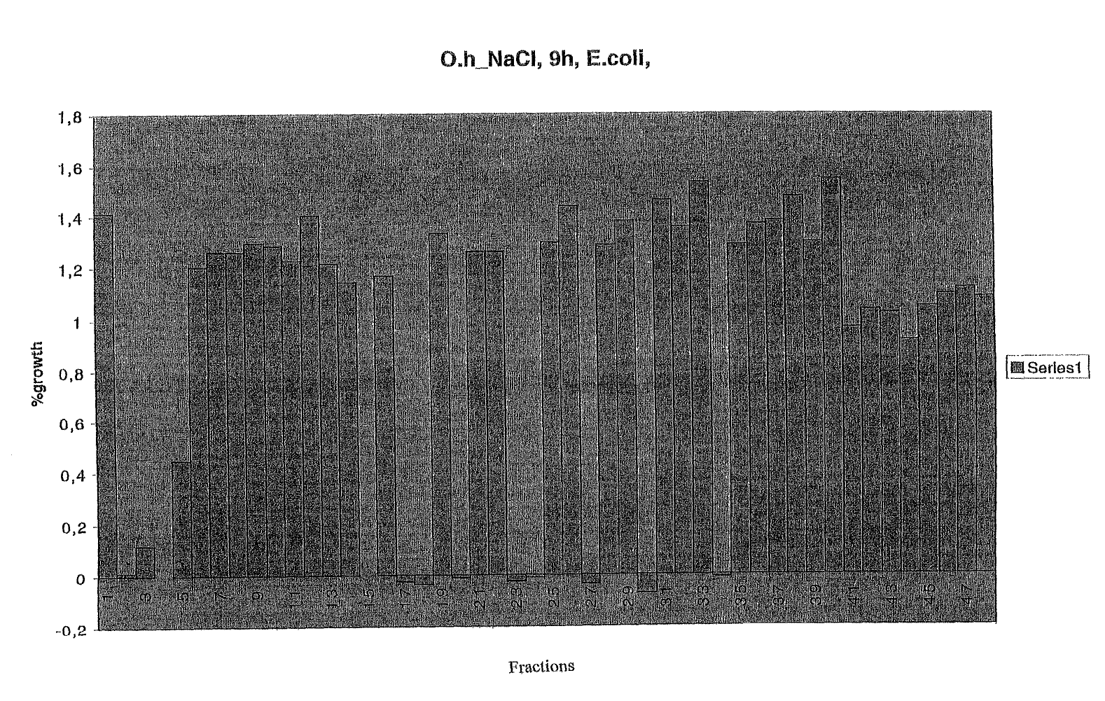

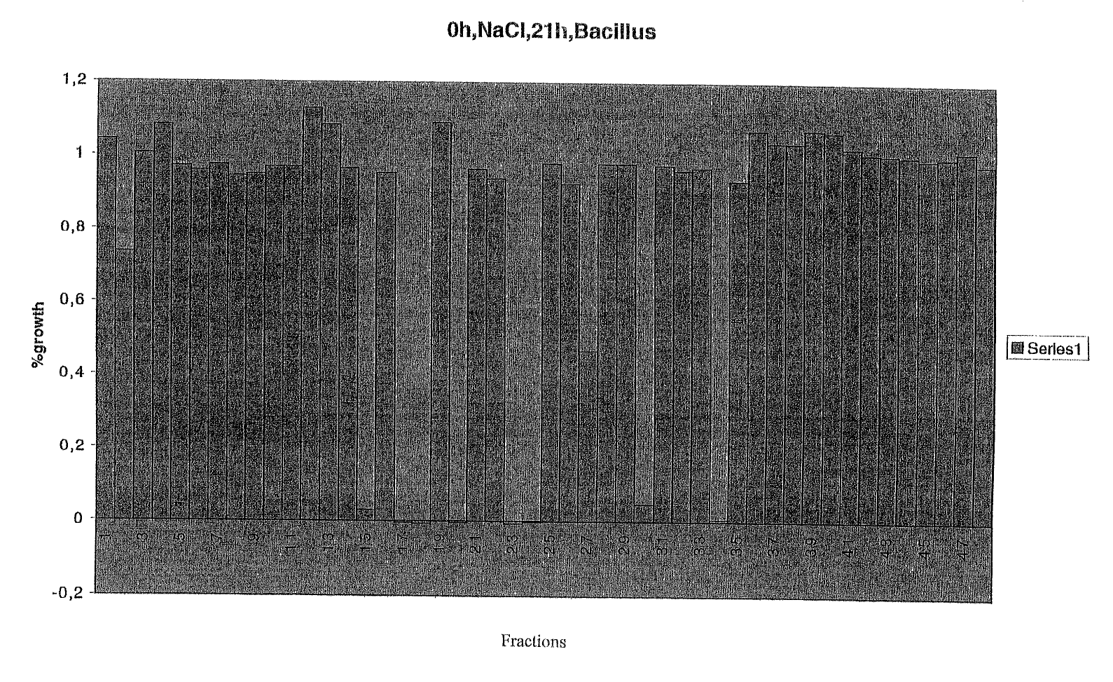

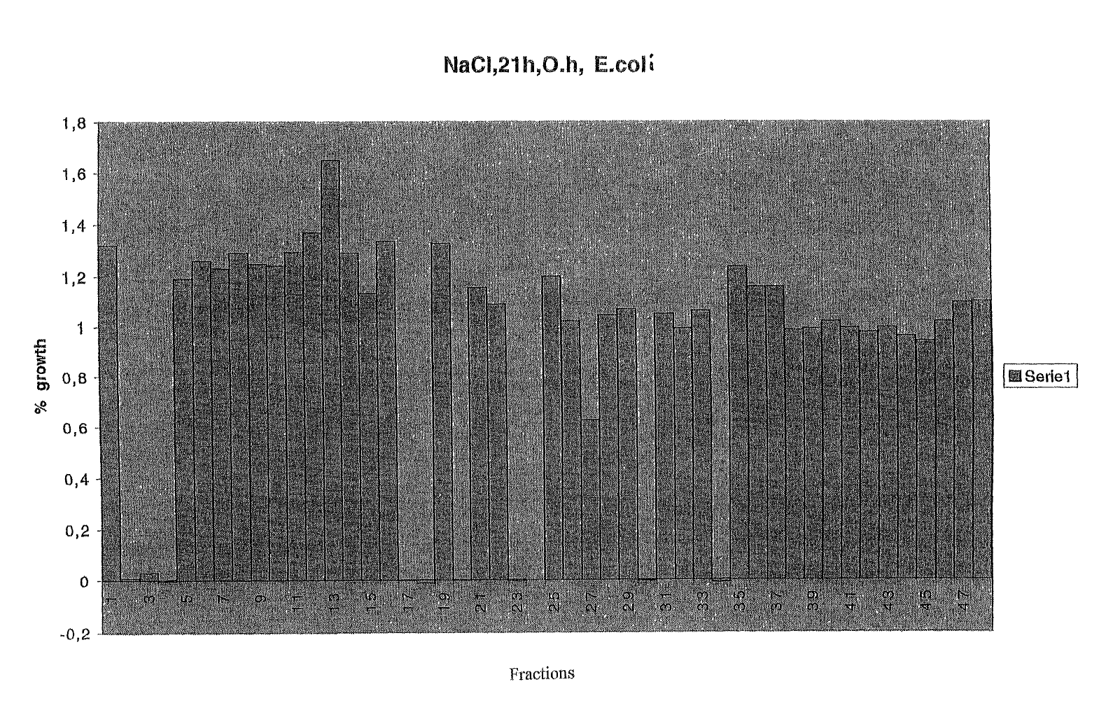

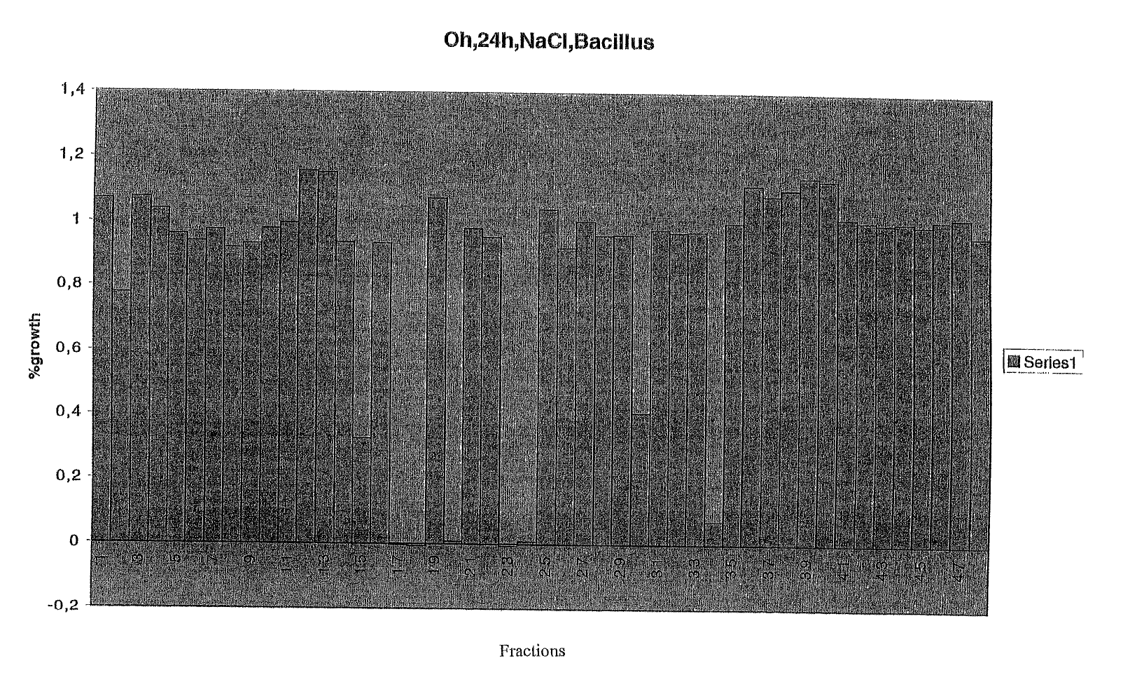

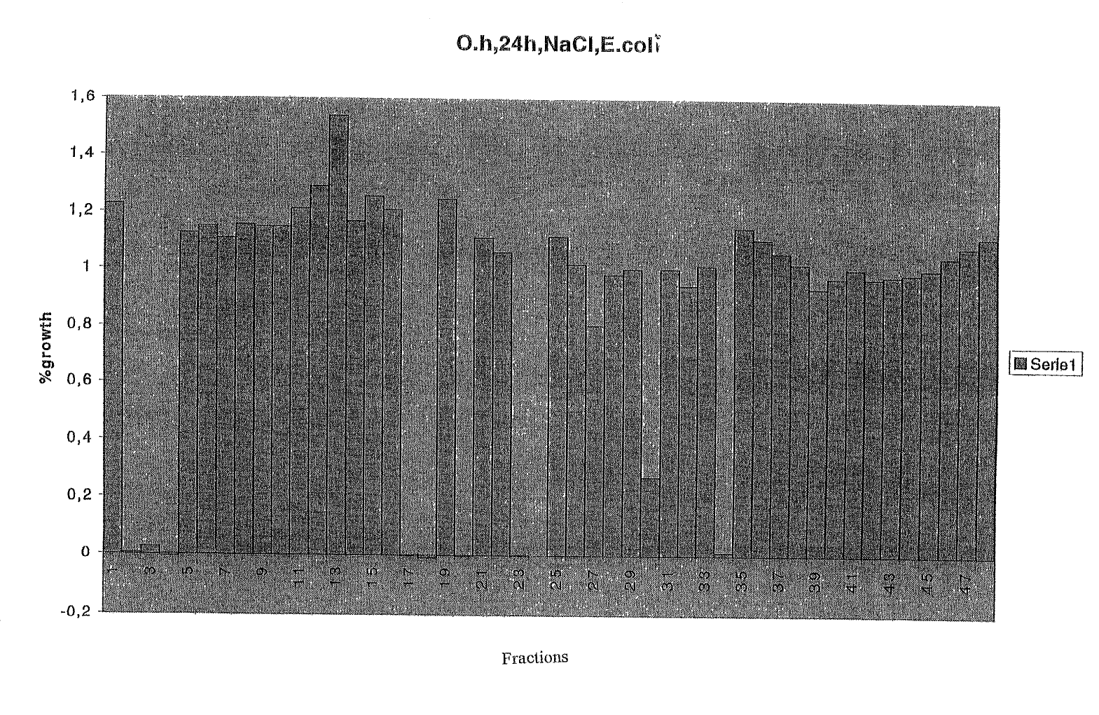

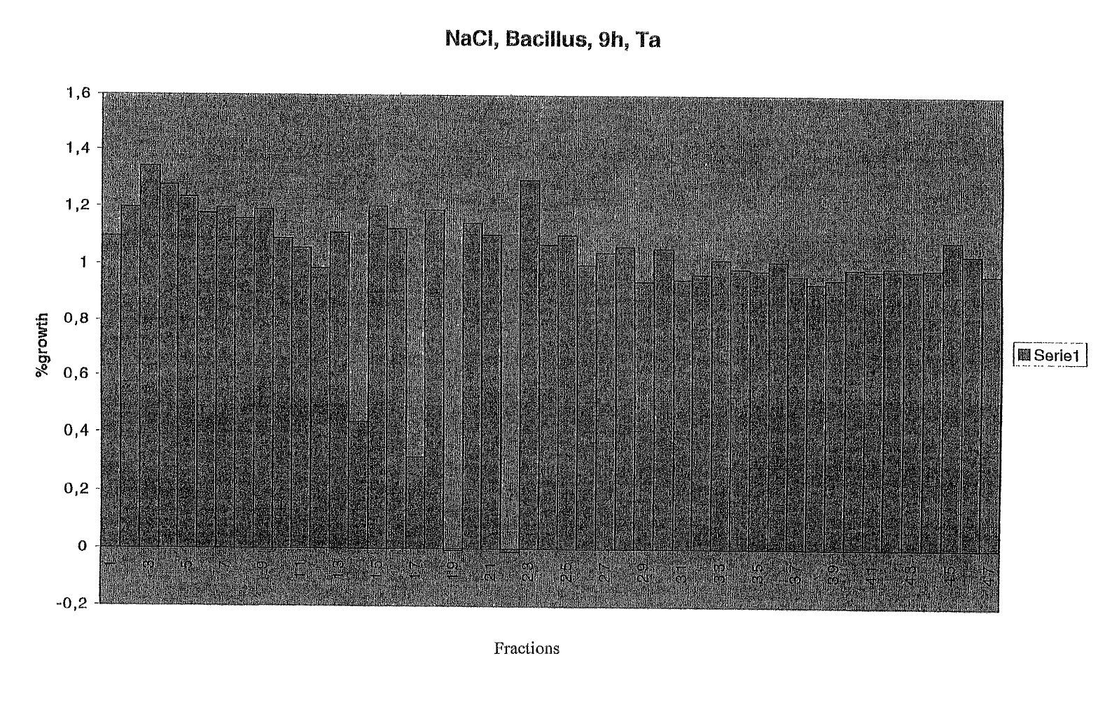

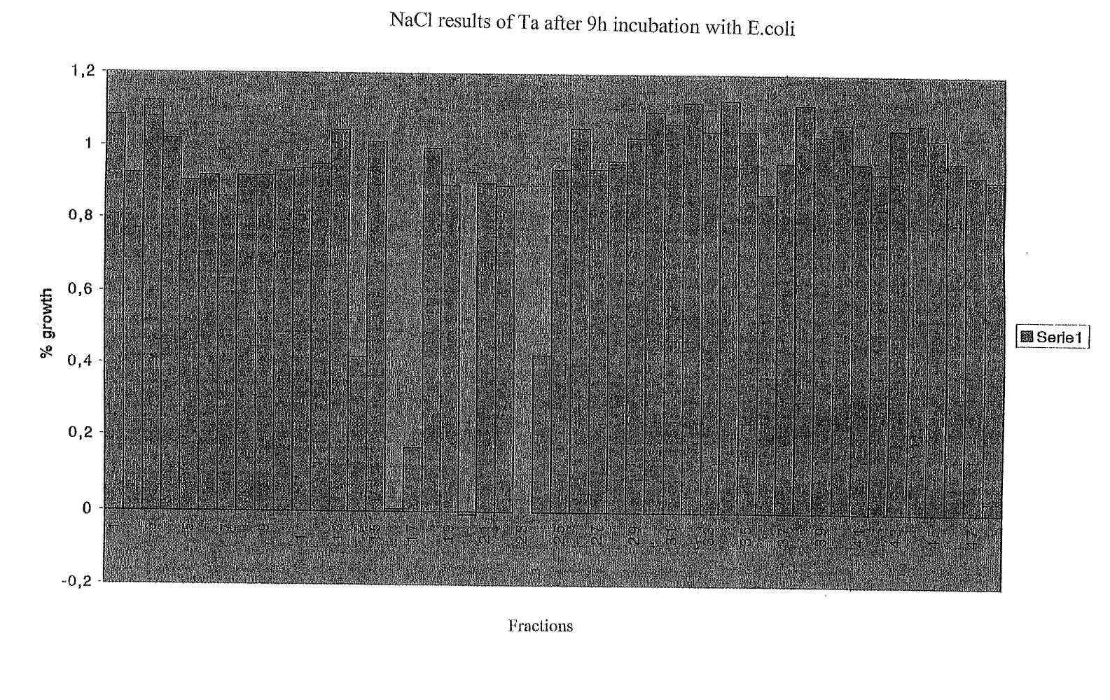

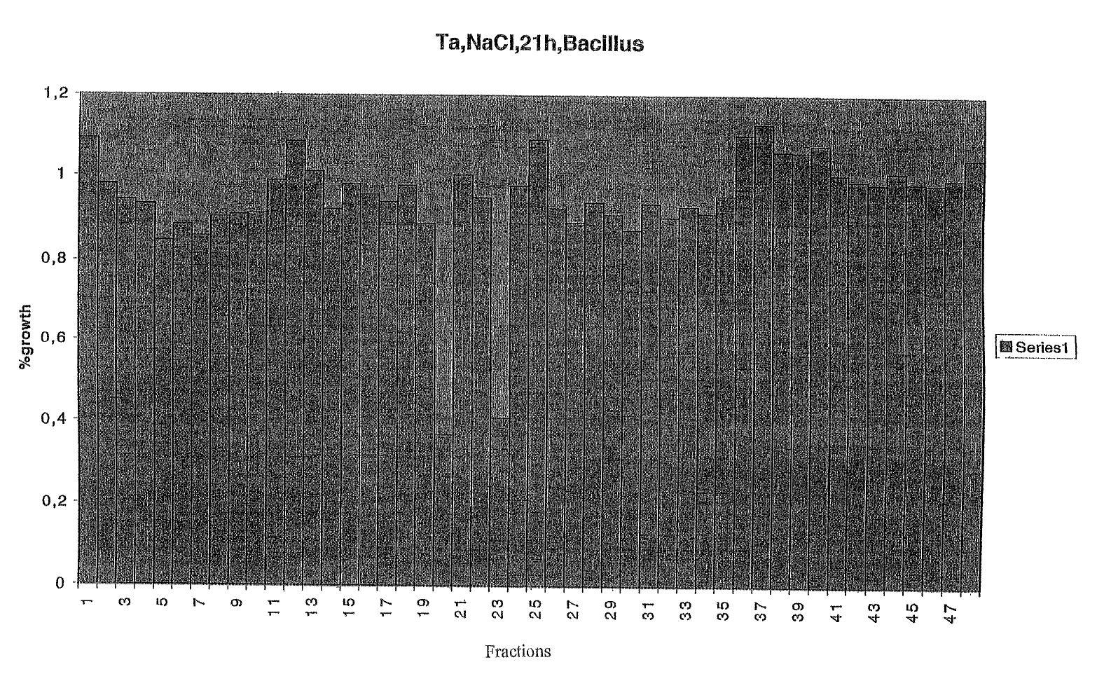

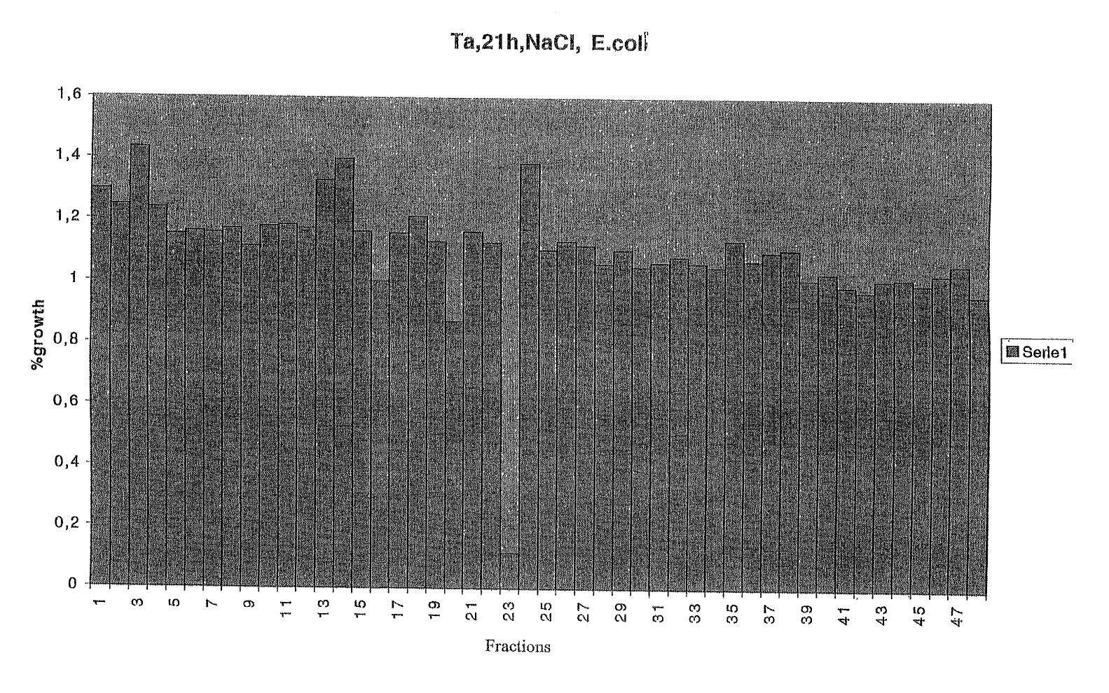

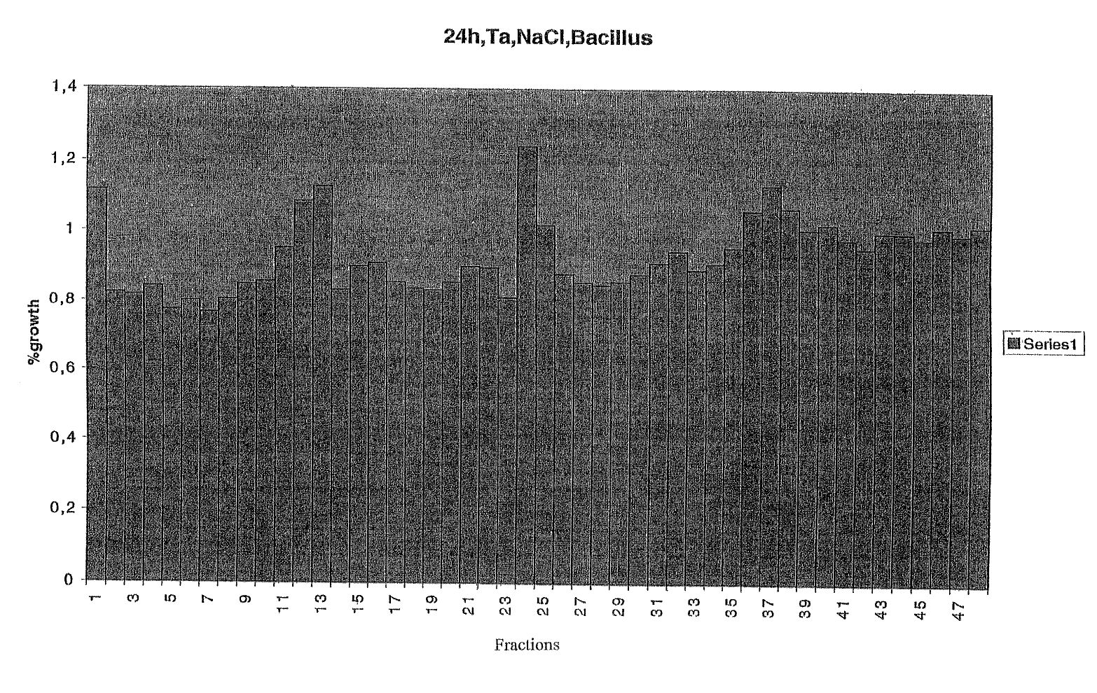

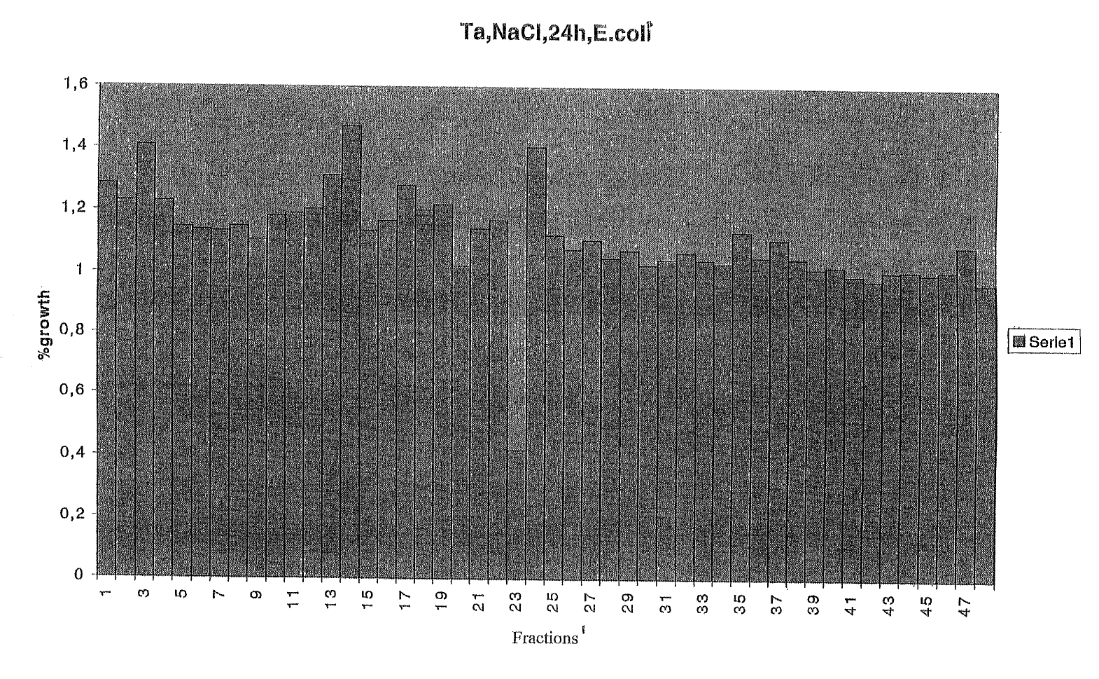

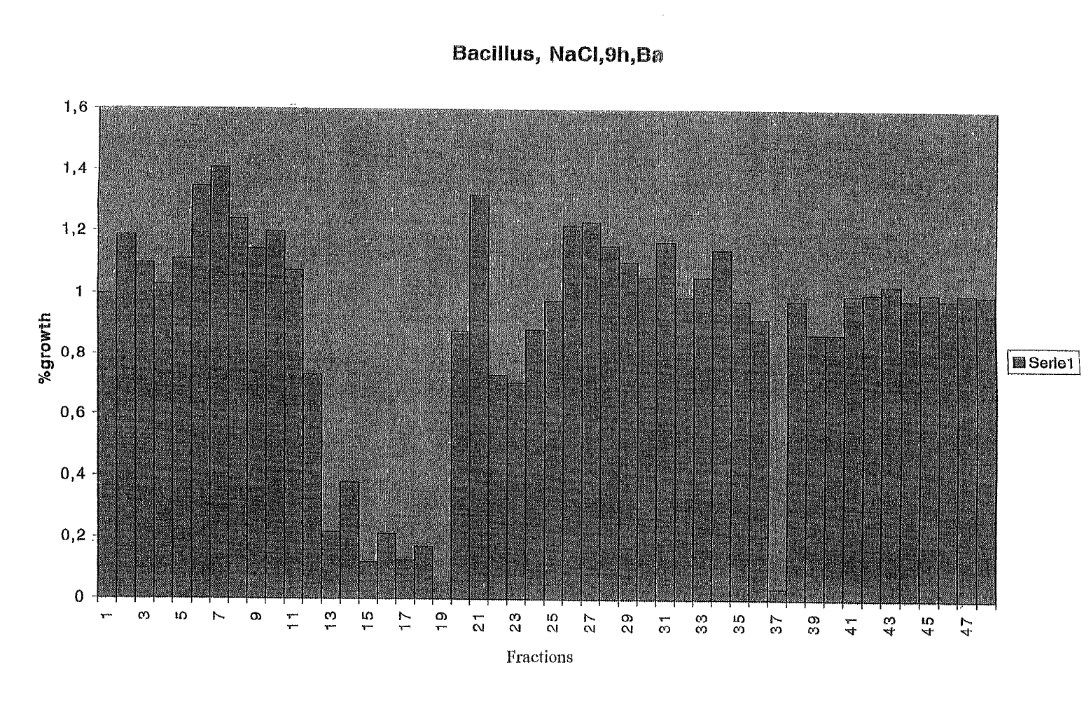

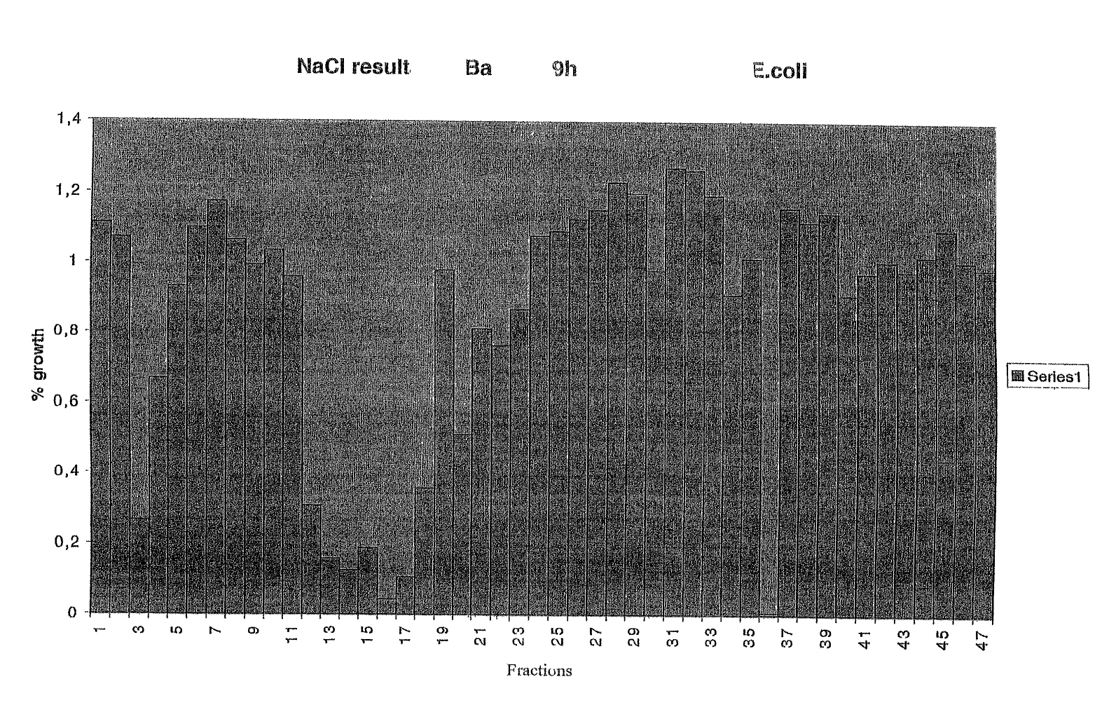

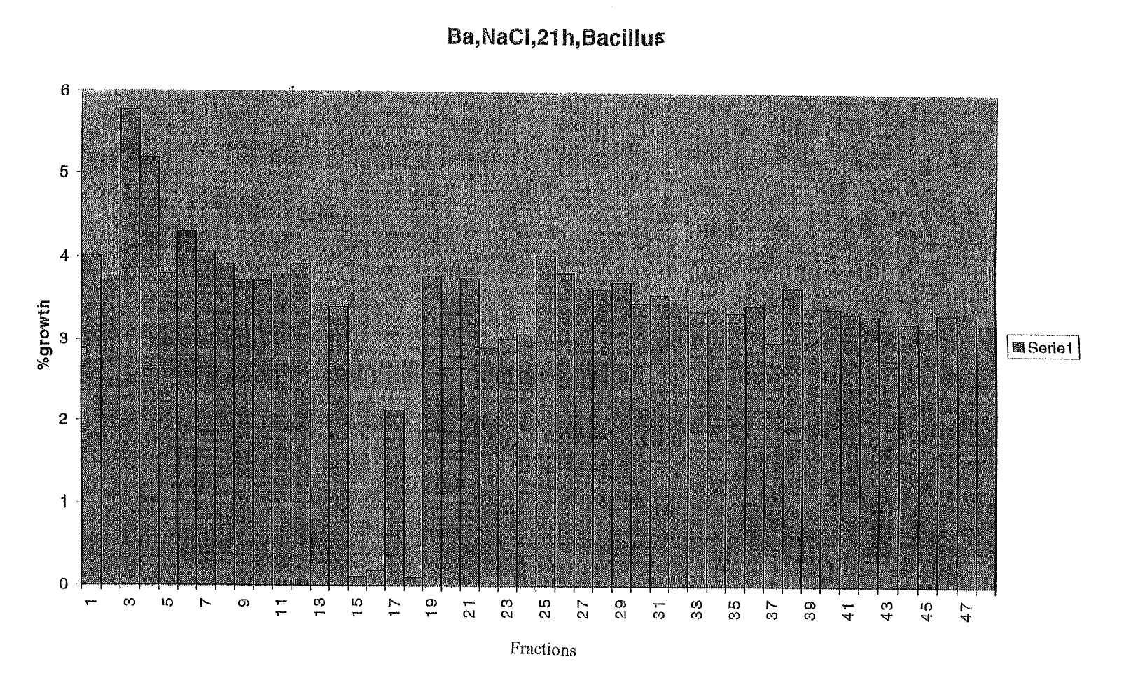

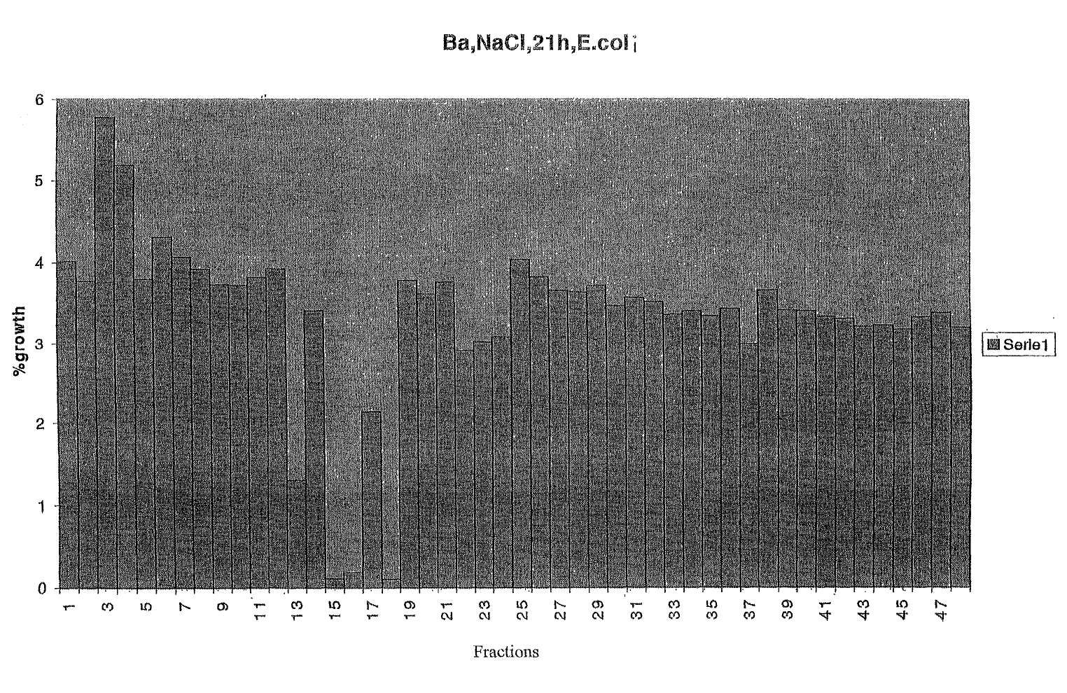

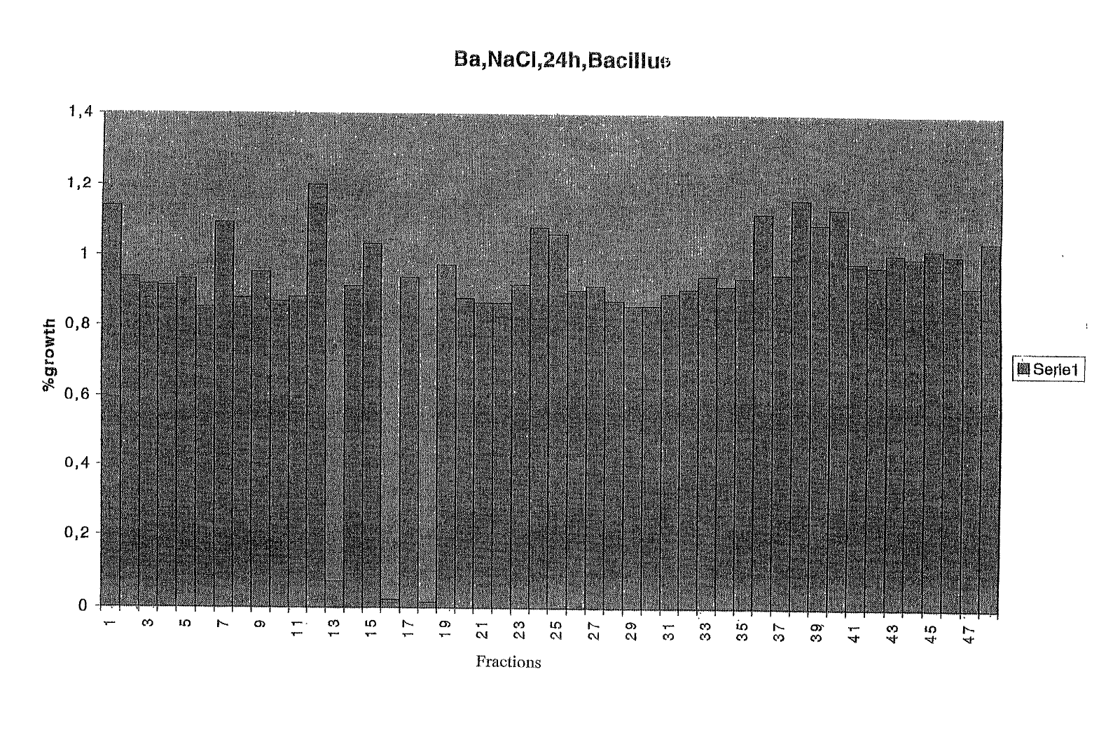

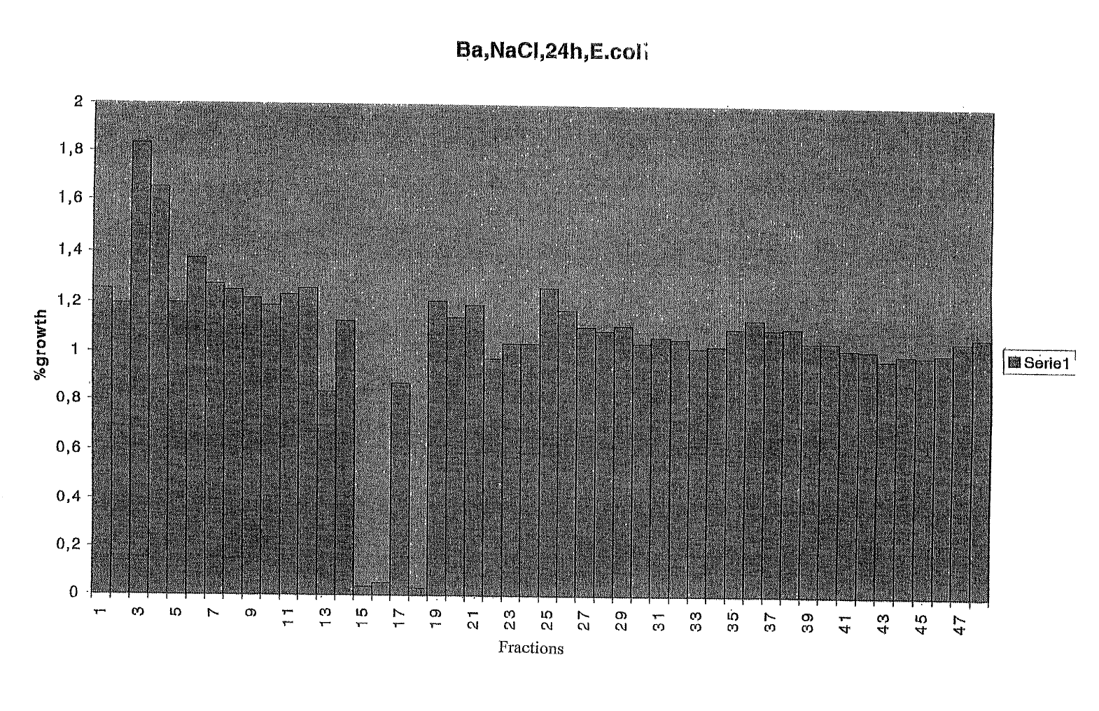

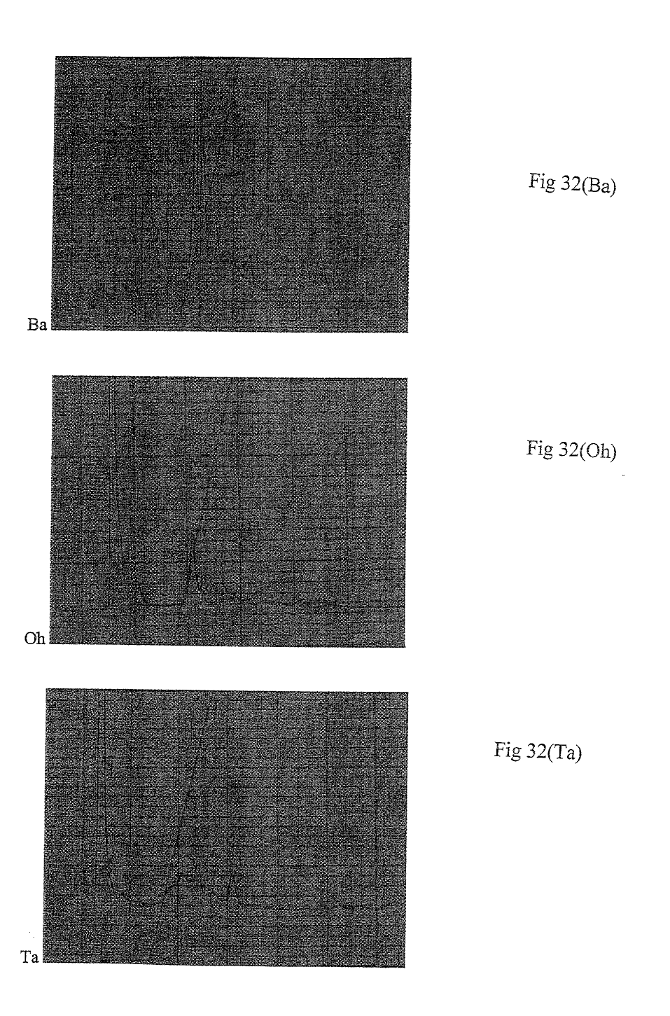

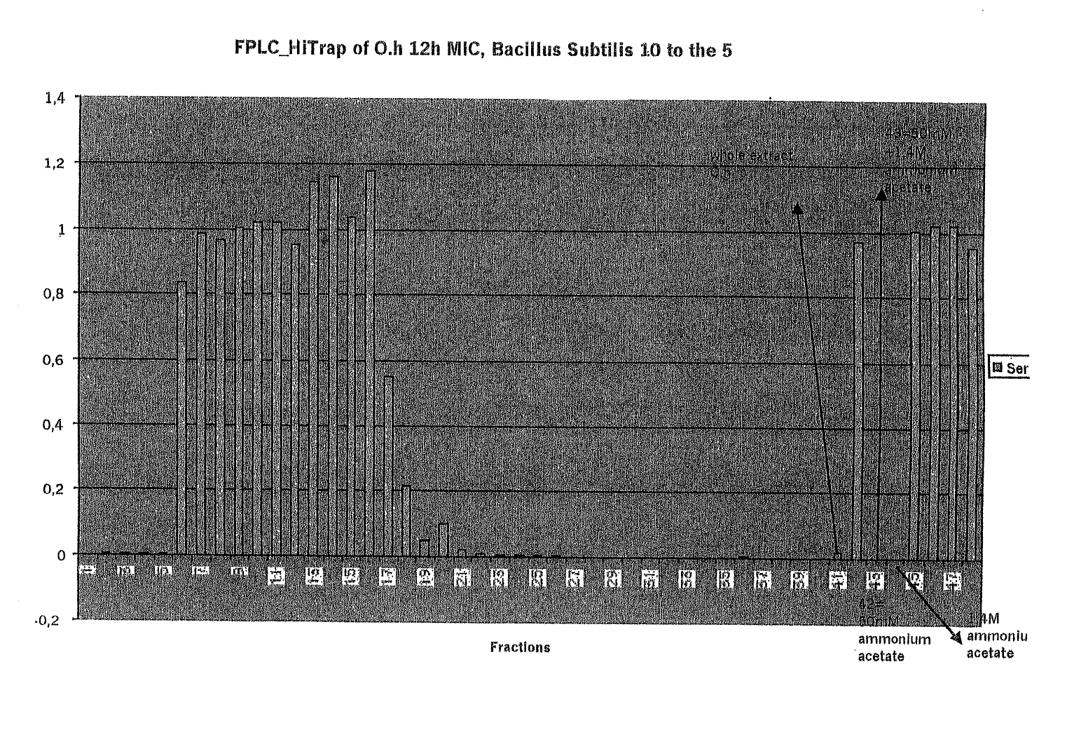

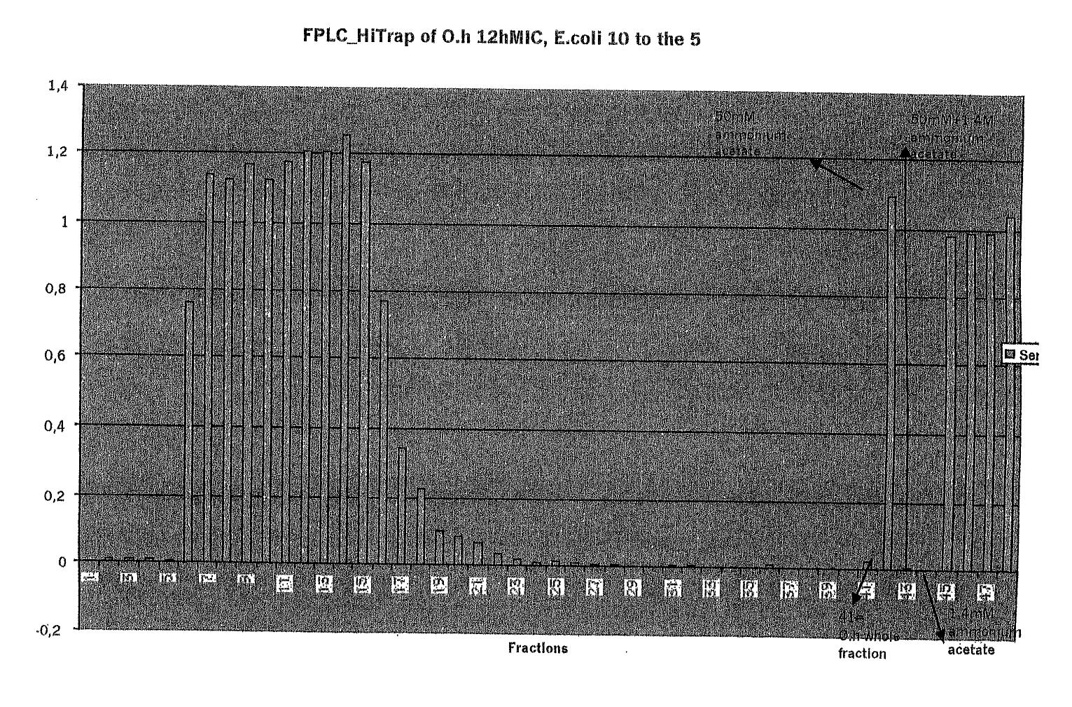

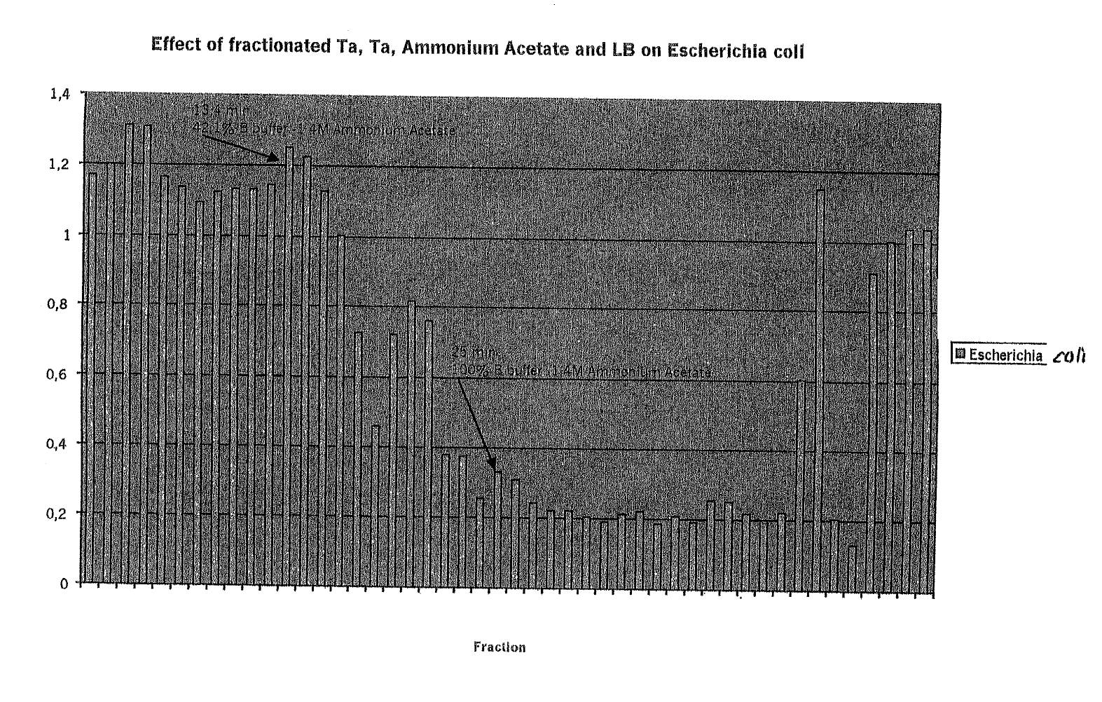

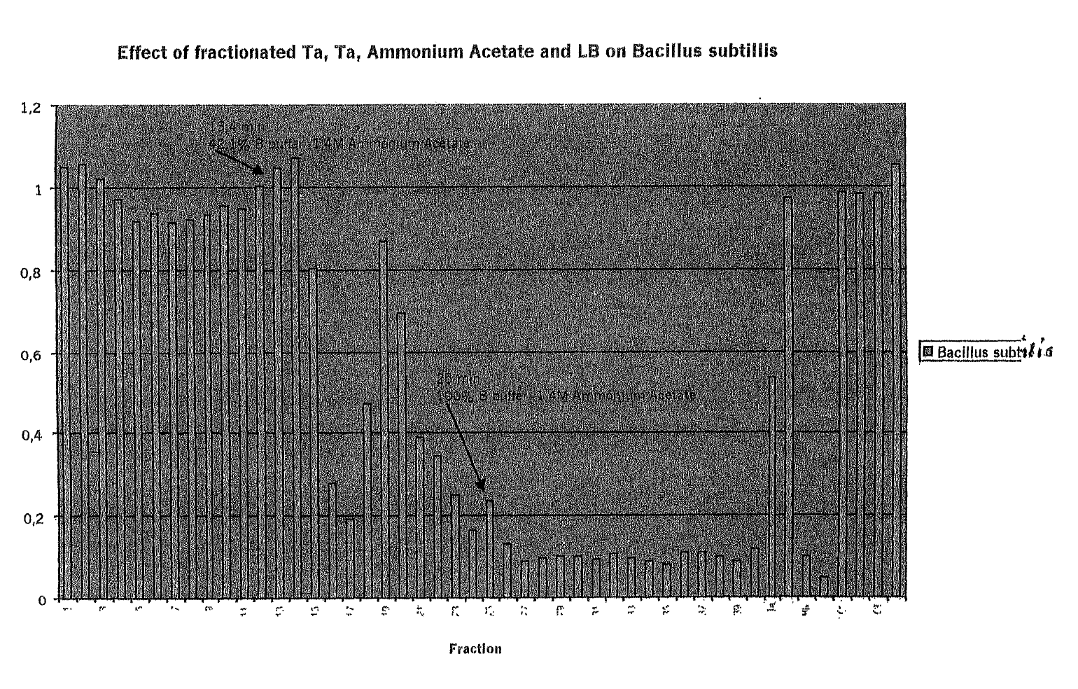

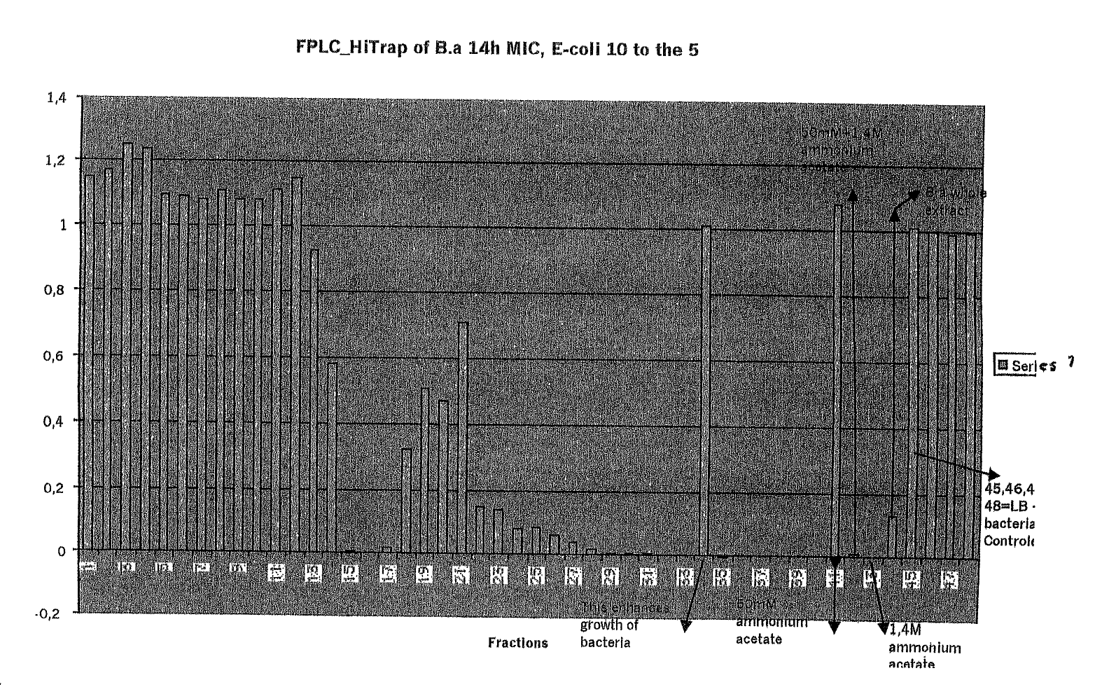

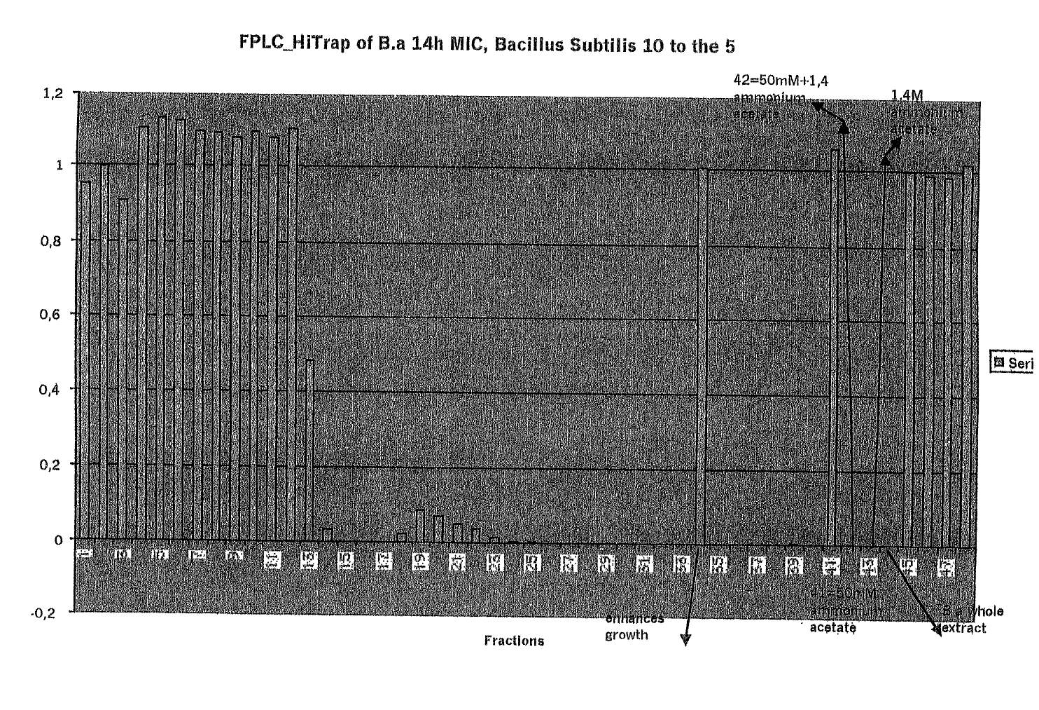

The resuspended fractions were used to treat bacterial cultures of Escherichia coli and Bacillus subtilis as follows; 60 μl of the fraction plus 40 μl of LB (Luria Broth) were added to 100 μl LB containing 10⁵ bacteria. Each experiment was made in duplicate and the effect on bacterial growth was determined by measuring O.D. (optical density) at 650 nm measured at 9, 21 and 24h for the minimal inhibitory concentration (MIC) . The results of this experiment 6 involving the above fractions when incubating with Bacillus and E. coli, respectively, are illustrated in FIG. 14-31 and in FIG. 33-38. When the peaks are approximately below the value 1 on the Y-axis, preferably below the value 0.9, on the Y-axis this indicates antibiotic effect of the peaks (which in turn are related to certain fractions of a venom, as outlined above). The fractions 41 to 48 shown in the form of peaks in the above figures mainly consisted of other substances than venom components (proteins) e.g. buffer and eluent. FIG. 32 shows the determination of protein content at 280nm as the fractions flowed out of the column for the respective venom BA, OH and TA (designated Ba, Oh and Ta, respectively, in the figure).

3. Peptide-protein pattern

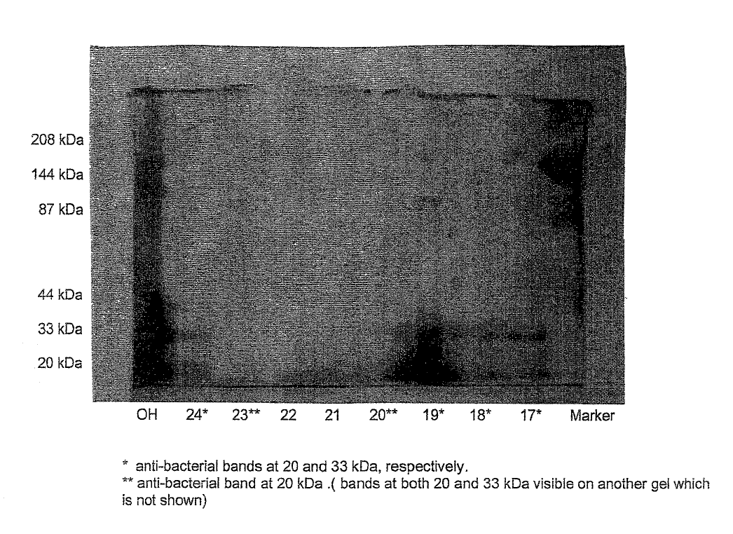

The protein content with regard to molecular weight of included peptid-protein components were studied in all FPLC-fractions from Oh, Ta and Ba. This was done using SDS-PAGE and subsequent staining of the proteins with silver (BIO-RAD Laboratories). We found that some of the anti-bacterial fractions contained peptid-protein bands not present in fractions without antibacterial effect. These bands are indicated in FIG. 44 (OH only). FIG. 44 shows a gel picture showing the protein content in selected FPLC-fractions from OH after SDS-polyacrylamide gel electrophoresis (SDS-PAGE, 7.5% PA) and silver staining. Protein bands appearing only in fractions with antibacterial activity are indicated with * or **. The standard marker which is depicted to right has the following weights in kDa: 208, 144, 87, 44, 33 and 20 kDa. The bands appearing in lanes 1 and 2 (i.e. fractions 17 and 18, respectively) thus are approximately 20 and 33 kDa. We found that several of the antibacterial fractions from OH (fractions 2, 3, 4, 15, 17, 18, 19, 20, 23 and 24) apparently contained only the same two peptid-protein bands, approximately 20 and 33 kDa, and these bands were not present in fractions without antibacterial effect. These bands are indicated in FIG. 44.

The protein content with regard to molecular weight of included peptid-protein components were studied in all FPLC-fractions from TA and BA also. This was done using SDS-PAGE (SDS-polyacrylamide gel electrophoresis) with 12% PA and subsequent staining of the proteins with silver (BIO-RAD Laboratories). For BA we identified a band, approximately at 30 kD, present as single in 2 of 4 active fractions (fractions 15 and 16) and these bands were not present in fractions without anti-bacterial effect.

4. Heat stability

The heat stability of antibiotic activity in selected fractions from OH, BA and TA was studied. The fractions were preincubated at 37° C. for 2, 4, 6, 8, 10 and 24 h before inoculation with bacteria. This was performed using the same protocol as in paragraph 2 above: “Antibiotic activity testing”, but with the following changes: The fractions were preincubated at 37° C. for 24 h before inoculation with bacteria. The bacterial growth was monitored after 2, 4, 6, 8, 10 and 24 h. The results suggested that preincubation at 37° C. for 24 h does not significantly reduce the antibacterial activity of any of the fractions.

Experiment 7

Toxicity tests

Epithelial cells 293T were cultured to confluence in monolayers. A serial dilution of the fractions was used to treat the cells and their capacity to reduce a metabolic marker (Alamar blue) and was compared to that of untreated cells. Such activity was examined by determining the colour change (blue to pink) in the metabolic marker at 490 and 650 nm. In addition microscopic evaluation of the cultures was performed at 17 hours post-treatment.

Results of the toxicity tests:

The results of the respective fractions are given in table 9 and 10 below:

TABLE 9
Results of the toxicity tests

| (ALSO FOR PLATE 1 | | | | | |
|---|---|---|---|---|---|
| READ AT | | (ALSO FOR | | | |
| OH14-20) 490 NM | | OH14-20) | | | |
| μg/ml | OH1 | OH2 | OH3 | OH4 | OH5 |
| 60 | 0.028 | −0.002 | −0.047 | −0.004 | 0.014 |
| 30 | 0.018 | −0.021 | −0.03 | −0.03 | 0.003 |
| 15 | 0.005 | −0.027 | −0.028 | −0.044 | −0.013 |
| 7.5 | −0.012 | 0.055 | 0.039 | 0.032 | 0.001 |
| 3.75 | −0.025 | −0.067 | −0.048 | −0.062 | −0.005 |
| 1.875 | −0.022 | −0.073 | −0.06 | −0.012 | 0.01 |
| 0.9375 | −0.021 | 0.007 | 0.005 | −0.006 | 0.012 |
| 0 | 0 | 0 | 0 | 0 | 0 |
| (ALSO FOR PLATE 2/ | | (ALSO FOR | | | |
| OH26-32) 490 | | OH26-32) | | | |
| μg/ml | OH21 | OH22 | OH23 | OH24 | OH25 |
| 60 | −0.032 | −0.024 | −0.036 | −0.054 | −0.048 |
| 30 | −0.008 | −0.003 | −0.032 | −0.048 | −0.024 |
| 15 | 0.002 | −0.004 | −0.043 | −0.042 | −0.022 |
| 7.5 | 0.001 | −0.005 | −0.05 | −0.034 | −0.015 |
| 3.75 | −0.003 | −0.022 | −0.049 | −0.03 | −0.028 |
| 1.875 | −0.02 | −0.024 | −0.012 | −0.029 | −0.035 |
| 0.9375 | −0.039 | 0.024 | −0.009 | −0.044 | −0.017 |
| 0 | 0 | 0 | 0 | 0 | 0 |
| (ALSO FOR PLATE 3/ | | (ALSO FOR | | | |
| TA14-20)490 | | TA14-20) | | | |
| μg/ml | OH33 | OH34 | OH35 | OH36 | TA13 |
| 60 | −0.056 | −0.059 | 0.013 | −0.044 | −0.033 |
| 30 | −0.042 | −0.042 | 0.018 | −0.022 | 0.006 |
| 15 | −0.027 | −0.042 | −0.007 | −0.02 | −0.002 |
| 7.5 | −0.039 | −0.027 | −0.01 | 0.006 | 0.013 |
| 3.75 | −0.02 | −0.032 | −0.001 | −0.005 | 0.0031 |
| 1.875 | −0.046 | −0.007 | 0.015 | −0.003 | 0.012 |
| 0.9375 | 0.005 | 0.005 | 0.031 | 0.005 | 0.018 |
| 0 | 0 | 0 | 0 | 0 | 0 |
| (ALSO FOR PLATE 4 / | | (ALSO FOR | | | |
| BA3-15) 490 | | BA3-15) | | | |
| μg/ml | TA21 | TA22 | TA23 | TA24 | BA2 |
| 60 | 0.011 | 0.047 | 0.042 | 0.014 | 0.053 |
| 30 | 0.024 | 0.054 | 0.054 | 0.015 | 0.039 |
| 7.5 | 0.026 | 0.051 | 0.065 | 0.031 | 0.06 |
| 1.875 | 0.019 | 0.043 | 0.06 | 0.031 | 0.052 |
| 3.73 | 0.039 | 0.044 | 0.069 | 0.033 | 0.047 |
| 1.873 | 0.028 | 0.025 | 0.058 | 0.033 | 0.046 |
| 0.9373 | 0.028 | 0.026 | 0.061 | 0.034 | 0.042 |
| 0 | 0 | 0 | 0 | 0 | 0 |
| (ALSO FOR PLATE 5 / | | (ALSO FOR | | | |
| BA21-con) 490 | | BA21-con) | | | |
| μg/ml | BA16 | BA17 | BA18 | BA19 | BA20 |
| 60 | 0.02 | 0.006 | 0.026 | 0.085 | 0.048 |
| 30 | −0.032 | 0.015 | 0.01 | 0.081 | 0.0131 |
| 15 | −0.042 | 0.019 | 0.04 | 0.079 | 0.012 |
| 7.5 | −0.044 | 0.008 | 0.015 | 0.07 | 0.001 |
| 3.75 | −0.029 | 0.023 | 0.032 | 0.076 | 0.006 |
| 1.875 | −0.019 | 0.014 | 0.03 | 0.08 | 0.006 |
| 0.9375 | 0.016 | 0.028 | 0.022 | 0.064 | −0.003 |
| 0 | 0 | 0 | 0 | 0 | 0 |

TABLE 10
Results of the toxicity tests (cont.)

| OH14 | OH15 | OH16 | OH17 | OH18 | OH19 | OH20 |
|---|---|---|---|---|---|---|
| 0.015 | 0.012 | −0.002 | −0.036 | −0.027 | −0.034 | −0.035 |
| 0.022 | 0.008 | 0.001 | −0.027 | −0.026 | −0.035 | −0.011 |
| 0.013 | 0.001 | −0.011 | −0.013 | −0.021 | −0.042 | −0.012 |
| 0.02 | 0.007 | 0.004 | −0.024 | −0.018 | −0.019 | 0.021 |
| 0.013 | 0.001 | −0.006 | −0.005 | −0.016 | −0.026 | 0.023 |
| 0.023 | 0 | 0.009 | 0.001 | −0.006 | −0.022 | −0.001 |
| 0.027 | 0.009 | 0.006 | 0 | −0.005 | −0.01 | 0.022 |
| 0 | 0 | 0 | 0 | 0 | 0 | 0 |
| OH26 | OH27 | OH28 | OH29 | OH30 | OH31 | OH32 |
| −0.042 | −0.042 | −0.005 | −0.055 | −0.022 | −0,049 | −0.011 |
| −0.011 | 0 | −0.038 | −0.053 | −0.014 | −0,021 | 0.003 |
| −0.016 | 0.002 | −0.035 | −0.039 | −0.017 | −0.021 | −0.007 |
| −0.011 | −0.004 | −0.024 | −0.032 | 0.002 | −0.01 | −0.005 |
| −0.009 | −0.001 | −0.007 | −0.043 | −0.004 | −0.007 | −0.0041 |
| −0.011 | −0.01 | −0.015 | −0.035 | −0.019 | −0.024 | −0.027 |
| −0.022 | −0.009 | −0.004 | −0.038 | −0.028 | −0.007 | −0.016 |
| 0 | 0 | 0 | 0 | 0 | 0 | |
| TA14 | TA15 | TA16 | TA17 | TA18 | TA19 | TA20 |
| −0.038 | −0.015 | −0.031 | −0.003 | −0.022 | −0.052 | −0.035 |
| −0.004 | 0.023 | −0.002 | −0.003 | −0.001 | −0.003 | −0.028 |
| 0.005 | 0.014 | 0.011 | 0.006 | −0.004 | −0.011 | —0.018 |
| −0.029 | 0.007 | 0.009 | 0.004 | −0.003 | −0.001 | −0.038 |
| −0.044 | −0.011 | −0.004 | −0.009 | −0.028 | −0.009 | −0.037 |
| −0.025 | 0.006 | 0.012 | 0.003 | −0.006 | −0.003 | −0.034 |
| 0.011 | 0.017 | 0.021 | 0.018 | 0.012 | 0.017 | −0.002 |
| 0 | 0 | 0 | 0 | 0 | 0 | 0 |
| BA3 | BA4 | BA11 | BA12 | BA13 | BA14 | BA15 |
| 0.029 | 0.035 | 0.067 | 0.039 | 0.038 | −0.005 | 0.063 |
| 0.012 | 0.022 | 0.046 | 0.015 | 0.057 | −0.015 | 0 |
| 0.05 | 0.061 | 0.052 | 0.022 | 0.053 | −0.019 | −0.005 |
| 0.032 | 0.052 | 0.036 | 0.027 | 0.063 | 0.035 | −0.017 |
| 0.043 | 0.06 | 0.058 | 0.04 | 0.047 | 0.02 | −0.014 |
| 0.038 | 0.053 | 0.051 | 0.03 | 0.048 | 0.022 | 0.021 |
| 0.046 | 0.054 | 0.037 | 0.027 | 0.042 | 0.017 | 0.042 |
| 0 | 0 | 0 | 0 | 0 | 0 | 0 |
| | | | | CON- | CON- | CON- |
| BA21 | BA35 | BA36 | BA37 | TROL | TROL | TROL |
| 0.047 | 0.067 | 0.029 | 0.051 | 0.071 | 0.091 | −0.006 |
| 0.016 | 0.048 | 0.012 | 0.021 | 0.022 | 0.028 | −0.013 |
| 0.021 | 0.042 | 0.017 | 0.017 | 0.011 | 0.01 | 0 |
| 0.018 | 0.03 | 0.009 | 0.007 | 0.005 | 0.007 | −0.003 |
| 0.017 | 0.043 | 0.024 | 0.025 | 0.01 | 0.014 | −0.005 |
| 0.014 | 0.033 | 0.009 | 0.027 | 0.005 | 0.005 | −0.033 |
| 0.019 | 0.035 | 0.006 | 0.017 | 0.009 | 0.005 | 0.014 |
| 0 | 0 | 0 | 0 | 0 | 0 | 0 |

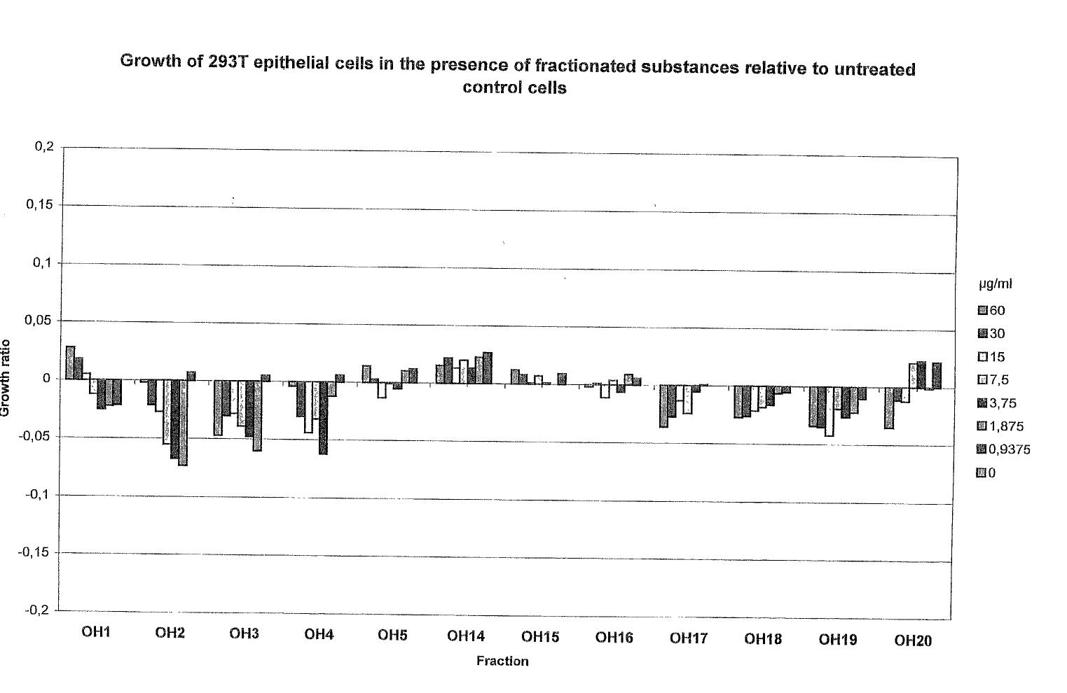

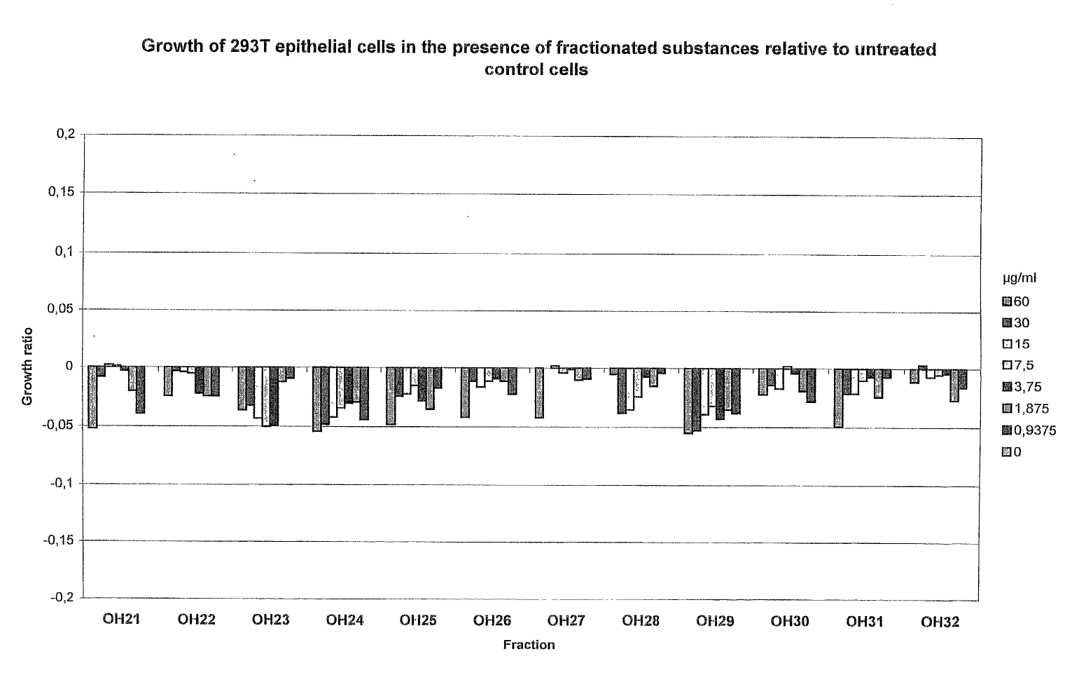

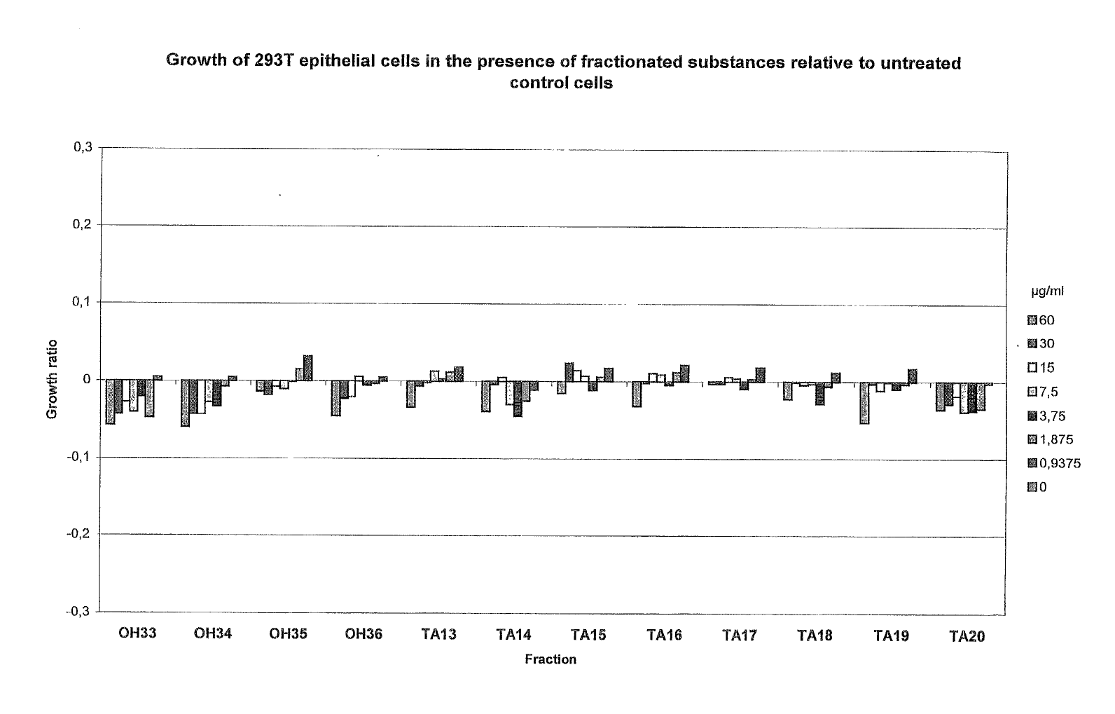

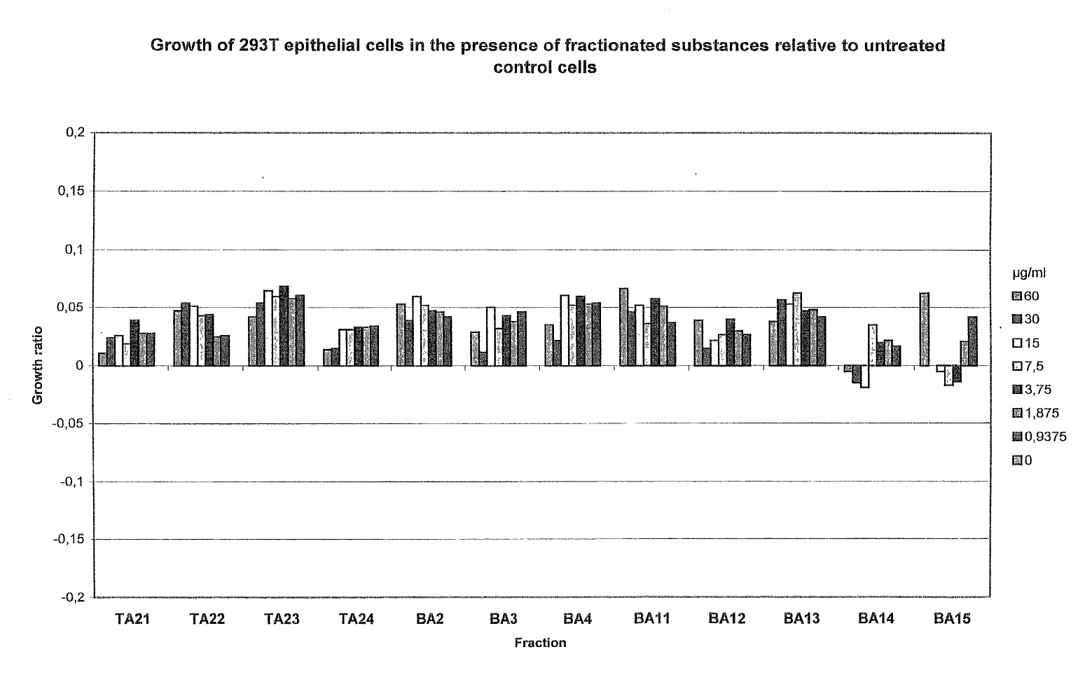

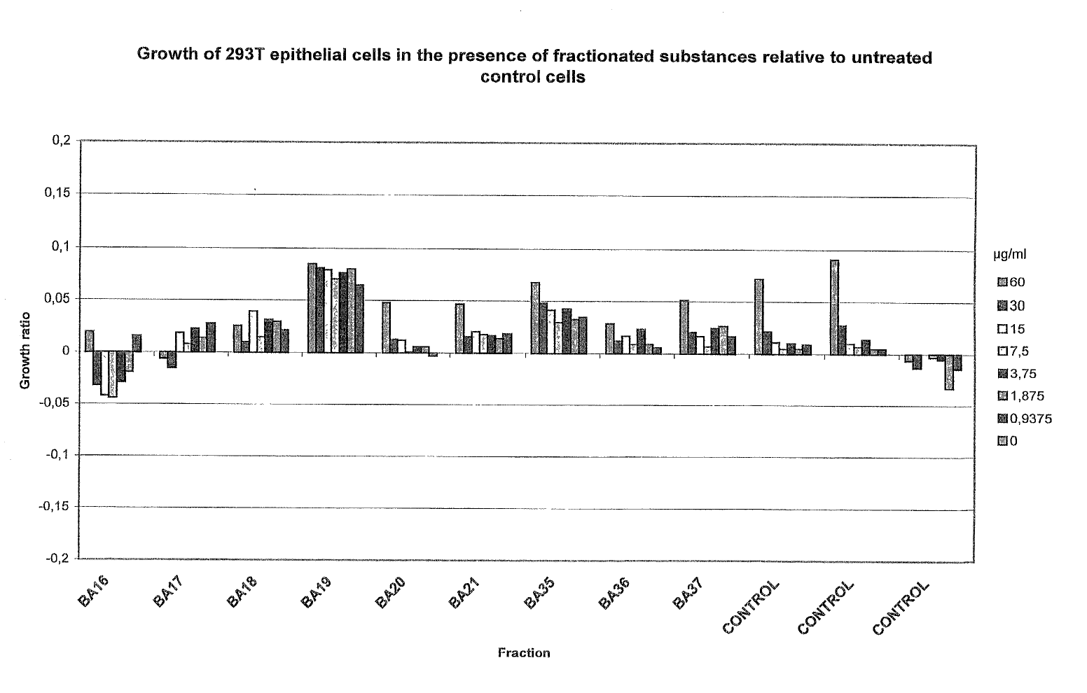

The activity of the marker was not affected by the venom fractions in a significant manner compared to untreated cells, after 17 hours of culture (time at which there were no additional colour change). Thus a metabolic activity apparently is present. However under microscopic examination there were changes compatible with cytopathic effect for 11/28 OH fractions, 7/12 TA fractions and 10/17 BA fractions. These results indicate that during the period of examination, the cell cultures retain their metabolic activity even though cytopathic changes might occur in a fraction of the population of treated cells. Results of the toxicity tests, given in tables 9 and 10, are illustrated in FIGS. 39-43.

Experiment 8: Viral tests

Inhibition assay

Inhibition of venoms on different virus was studied. Inhibition of stem, leaf and root extract from Aconitus on different virus was also studied.

Materials:

Substances to be tested for viral inhibitory effect:

1. Stem, leaves and root from Aconitus.

The pieces were placed in a double volume of phosphate buffered saline (PBS) at 4° C. over night. The extracts were subsequently filtered through Whatman filter and finally sterile filtered through a Whatman filter and finally sterile filtered through a 45 micron filter.

2. Freeze dried substances (venoms) from NK, BF, TA and BA. 500 μg of each venom was dissolved in 10 ml of PBS.

The portions were aliquoted and stored at −20° C.

Cell cultures:

Green Monkey kidney cells (VERO) deriving from the American Type Culture Collection (ATCC).

Viruses:

Coxsackie B2 and Sendai virus strains were derived from the Swedish institute for Infectious Disease control. Viruses were titrated to yield 100 infectious doses (ID50) per 0.1 ml at inoculation of the VERO cells. The viruses are both RNA viruses, for which there are currently no antiviral drugs. Coxsackie B2 is a picorna virus, a non-enveloped virus. Sendai virus is a paramyxovirus, an enveloped virus. They can be considered as model viruses. Herpes Simplex virus type 1 was also used.

Results:

Toxicity tests

Toxicity was evaulated by light microscopy for one week. Venom BA was toxic to cells down to dilution of 1/100 000, followed by venom TA at 1/10000. The plant extracts were only toxic to the cells down to 1/10. The substances were therefore used at a tenfold lower dilution than the least toxic dilution.

Virus inhibition tests

These were done three times. The outcome of the inhibitions were judged by microscopy after 1 week. Absence of viral cytopathic effect (CPE) and absence of signs of cellular toxicity, while the inhibited virus control showed clear CPE, was judged as a viral inhibition.

Coxsackie B2 essentially never got inhibited by any substance. However its own CPE got enhanced by the venoms. The unexpected enhancing effect could be diluted at least 1 million times. Thus it gave evidence for a highly specific interaction of virus and substance.

Sendai virus was inhibited by venoms NK and BF, at dilutions of 1/10000 and 1/1000, respectively. It was essentially inhibited by non-toxic concentrations of venoms TA and BA.

Herpes simplex virus type 1 was inhibited by BF at a further dilution of 1/10.

Conclusions

There was an inhibition of Sendai virus by venoms NK and BF. The infectivity enhancement of Cocksackie B2 virus was pronounced. Such interactions may be converted to inhibition once their mechanism is clarified. There was an inhibition of Herpes simplex virus type 1 by the venom BF, which suggests that BF venom or a component thereof could be useful for treating HIV as there is a pronounced similarity between the herpes simplex virus and the HIV virus

While the invention has been described in terms of various preferred embodiments, the skilled artisan will appreciate that various modifications, substitutions, omissions and changes can be made without departing from the spirit thereof. Accordingly, it is intended that the scope of the present invention be limited solely by the scope of the following claims, including equivalents thereof.

1. A pharmaceutical composition comprising an antimicrobially effective amount of at least one snake venom obtainable from a snake selected from the group of snakes consisting of: Bungarus fasciatus, Ophiophagus hannah, Bungarus candidus, Lapemis hardwickii, Hydrophis cyanocinctus, Enhydrina schistosa, Aipysurus eydouxii, Bothrops atrox, Lachesis muta, Bitis arietans, Trimersurus albolabris or combinations thereof; and a pharmaceutically acceptable carrier.

2. A pharmaceutical composition according to claim 1 comprising snake venom obtainable from a snake selected from the group of snakes consisting of: Bungarus fasciatus, Ophiophagus hannah, Bungarus candidus, Bitis arietans or Trimersurus albolabris; and a pharmaceutically acceptable carrier.

3. A pharmaceutical composition according to claim 1 or 2 comprising snake venom obtainable from Ophiophagus hannah and a pharmaceutically acceptable carrier.

4. A pharmaceutical composition according to claim 1 or 2 comprising snake venom obtainable from Bungarus fasciatus and a pharmaceutically acceptable carrier.

5. A pharmaceutical composition according to claim 1 or 2 comprising snake venom obtainable from Bitis arietans and a pharmaceutically acceptable carrier.

6. A pharmaceutical composition according to claim 1 or 2 comprising snake venom obtainable from Trimersurus albolabris and a pharmaceutically acceptable carrier.

7. A pharmaceutical composition according to any of claims 1 to 3 comprising three snake venoms obtainable from Naja kaouthia, Bungarus fasciatus and Ophiophagus hannah, respectively and a pharmaceutically acceptable carrier.

8. A pharmaceutical composition according to claim 1 or 2 comprising at least two snake venoms from two different groups of snakes, wherein the first venom is influencing the human nervous system and the second venom is influencing the human muscular system; and a pharmaceutically acceptable carrier.

9. A pharmaceutical composition according to claim 8, wherein the first venom is obtainable from a snake selected from the group of snakes consisting of: Naja kaouthia, Bungarus fasciatus, Ophiophagus hannah and Bungarus candidus; and the second venom is obtainable from a snake selected from the group of snakes consisting of: Lapemis hardwickii, Hydrophis cyanocinctus, Enhydrina schistosa, Aipysurus eydouxii, Bothrops atrox, Lachesis muta, Bitis arietans or Trimersurus albolabris; and a pharmaceutically acceptable carrier.

10. A pharmaceutical composition according to claim 9, wherein the first venom is obtainable from Ophiophagus hannah and the second venom is obtainable from Bitis arietans or Trimersurus albolabris.

11. A method for purification of at least one pharmaceutically active fraction of a snake venom obtainable from a snake comprised in the group Ophiophagus hannah, Bitis arietans and Trimersurus albolabris, comprising the steps:
a) dissolving venom with one or more suitable agents to a final concentration of approximately 0.01 mM;
b) passing the dissolved venom diluted, approximately 1/10, through an ion exchange cationic column with approximately 1 ml capacity with a flow of approximately 0.5 ml/min; and
c) recovery of 40 fractions of approximately 600 μl each.

12. A method according to claim 11 wherein the fractions of step c) are:
for Ophiophagus hannah (OH) : No. 1, 2, 3, 4, 5, 6, 7, 8, 12, 15, 16, 17, 18, 19, 20, 21, 22, 23, 24, 25, 26, 27, 28, 29, 30, 31, 32, 33, 34, 35, 36, 37, 38, 39, 40; preferably No. 2, 3, 4, 15, 17, 18, 19, 20, 23, 24, 30, 33, 34, 35 and 36; most preferred No. 2, 3, 4, 15, 17, 18, 19, 20, 23, 24, 33, 34, 35, 36;
for Trimersurus albolabris (TA) : No. 2, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 and 40; preferably No. 13, 14, 15, 16, 17, 18, 19, 20, 21, 22, 23 and 24; most preferred No. 13, 14, 15, 16, 17, 18, 19, 20, 21, 22, 23 and 24; and
for Bitis arietans (BA) : No. 2, 3, 4, 5, 6, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 and 40; preferably No. 3, 12, 13, 14, 15, 16, 17, 18, 19, 22, 23, 36 and 37; most preferred No. 2, 3, 4, 11, 12, 13, 14, 15, 16, 17, 18, 19, 20, 21, 35, 36 and 37.

13. A fraction of a venom obtainable by a method according to any one of claims 11 or 12.

14. A pharmaceutical composition comprising a fraction of a venom according to claim 13 and a pharmaceutically acceptable carrier.

15. A pharmaceutical composition comprising a component of a venom obtainable from Ophiophagus hannah, Bitis arietans or Trimersurus albolabris wherein the weight of the component is from approximately 20 to approximately 33 kDa; and a pharmaceutically acceptable carrier.

16. A pharmaceutical composition according to claim 15 comprising a component of a venom obtainable from Ophiophagus hannah wherein the weight of the component is approximately 20 or 33 kDa; and a pharmaceutically acceptable carrier.

17. A pharmaceutical composition according to claim 16 comprising two components of a venom obtainable from Ophiophagus hannah wherein the weights of the components are approximately 20 and 33 kDa, respectively; and a pharmaceutically acceptable carrier.

18. A pharmaceutical composition according to claim 15 comprising a component of a venom obtainable from Bitis arietans wherein the weight of the component is approximately 30 kDa; and a pharmaceutically acceptable carrier.

19. A pharmaceutical composition according to any of claims 1 to 6 comprising at least one snake venom and at least one plant extract, preferably obtainable from the plant family Ranunculaceae; and a pharmaceutically acceptable carrier.

20. A pharmaceutical composition according to claim 19 comprising snake venom from Bitis arietans or Trimersurus albolabris and at least one plant extract; and a pharmaceutically acceptable carrier.

21. A pharmaceutical composition according to claim 8 comprising at least two snake venoms from two different groups of snakes and at least one plant extract; and a pharmaceutically acceptable carrier.

22. A pharmaceutical composition according to any of claims 19 to 20 wherein the plant extract is obtainable from the plant Aconitum septentrionale, Aconitum karacolicum or Aconitum napellus.

23. A pharmaceutical composition according to claim 19 wherein the plant extract is aconitine.

24. A pharmaceutical composition according to any of claims 1 to 11 or any of claims 14 to 23, adapted for oral administration.

25. A snake venom, a fraction thereof or a component thereof obtainable from a snake comprised in the group Bungarus fasciatus, Ophiophagus hannah and Bungarus candidus, Lapemis hardwickii, Hydrophis cyanocinctus, Enhydrina schistosa, Aipysurus eydouxii, Bothrops atrox, Lachesis muta, Bitis arietans or Trimersurus albolabris for medical use.

26. Therapeutic use of a snake venom, a fraction thereof or a component thereof obtainable from a snake comprised in the group Bungarus fasciatus, Ophiophagus hannah and Bungarus candidus, Lapemis hardwickii, Hydrophis cyanocinctus, Enhydrina schistosa, Aipysurus eydouxii, Bothrops atrox, Lachesis muta, Bitis arietans or Trimersurus albolabris.

27. The use of a snake venom, a fraction thereof or a component thereof obtainable from a snake comprised in the group Naja kaouthia, Bungarus fasciatus, Ophiophagus hannah and Bungarus candidus, Lapemis hardwickii, Hydrophis cyanocinctus, Enhydrina schistosa, Aipysurus eydouxii, Bothrops atrox, Lachesis muta, Bitis arietans, Trimersurus albolabris or aconitine in the manufacture of a medicament for the prevention, management or treatment of bacterial, fungal, protozoan or viral diseases.
